(19) United States
(12) Patent Application Publication
(10) Pub. No.: US 2026/0347780 A1
(43) Pub. Date: Nov. 5, 2026

(54) ISOGENIC CARDIAC ORGANOIDS AND METHODS OF MAKING AND USING THE SAME

(71) Applicants: Ying Mei, Mount Pleasant, SC (US); Dimitrios C. Arhontoulis, Charleston, SC (US); Charles M. Kerr, Charleston, SC (US); Sophia Silver, Mount Pleasant, SC (US)

(72) Inventors: Ying Mei, Mount Pleasant, SC (US); Dimitrios C. Arhontoulis, Charleston, SC (US); Charles M. Kerr, Charleston, SC (US); Sophia Silver, Mount Pleasant, SC (US)

(21) Appl. No.: 19/640,869

(22) Filed: Apr. 7, 2026

Related U.S. Application Data
(60) Provisional application No. 63/785,230, filed on Apr. 8, 2025.

(51) Int. Cl. C12N 5/077; A61K 35/34; C12N 5/071; C12N 5/0775

(52) U.S. Cl. CPC C12N 5/0657; A61K 35/34; C12N 5/0668; C12N 5/069; C12N 2506/45; C12N 2513/00

(57) ABSTRACT

This invention relates to relates to isogenic three-dimensional (3D) cardiac organoids, such as those comprising four different cardiac cell types that comprise cardiomyocytes (CMs) and nonmyocytes, and methods of making and using the same. Cardiomyocytes and nonmyocytes included in an isogenic 3D cardiac organoid described herein may be derived from (i.e., source) isogenic pluripotent stem cells (iPSCs).

### STATEMENT OF PRIORITY

This application claims the benefit, under 35 U.S.C. § 119(e), of U.S. Provisional Application No. 63/785,230 filed on Apr. 8, 2025, the entire contents of which are incorporated by reference herein.

### STATEMENT OF GOVERNMENT SUPPORT

This invention was made with government support under Grant Number HL173532, HL169361, and HL175050 awarded by the National Institutes of Health. The government has certain rights in the invention.

### FIELD OF THE INVENTION

This invention relates to isogenic three-dimensional (3D) cardiac organoids, such as those comprising four different cardiac cell types that comprise cardiomyocytes (CMs) and nonmyocytes, and methods of making and using the same.

### BACKGROUND OF THE INVENTION

Human pluripotent stem cell-derived cardiomyocytes (hPSC-CMs) have emerged as a favorable cell source for cardiac regenerative therapies. Dissociated hPSC-CMs delivered via intramyocardial injection have demonstrated the potential for functional recovery in injured hearts of mice, rats, guinea pigs, pigs, and non-human primates. However, injection of cell suspensions of hPSC-CMs often results in poor engraftment rates due to low cell survival accompanied by anoikis-mediated cell death. Tissue engineered constructs in the form of cardiac patches have been developed to mitigate anoikis-mediated cell death and improve graft retention. However, these cardiac patches are limited by fibrotic encapsulation and the inability to electromechanically couple with host tissue. Additionally, their transplantation involves invasive suturing onto the heart, putting vulnerable patients at additional risk.

Accordingly, solutions are needed for regenerating heart tissue that facilitate interactions between cells and surrounding extracellular matrix to improve graft retention following transplantation and that can be comprised of multiple cell types to allow for pre-vascularization of microtissues and creation of constructs larger than the oxygen diffusion limit of ~150 μm.

### SUMMARY OF THE INVENTION

A first aspect of the present invention is directed to an isogenic three-dimensional (3D) cardiac organoid, comprising four different cardiac cell types that comprise cardiomyocytes (CMs) and nonmyocytes, wherein the cardiomyocytes and nonmyocytes are derived from (i.e., source) isogenic pluripotent stem cells (iPSCs).

An additional aspect of the present invention is directed to a method of making an isogenic 3D cardiac organoid, the method comprising: culturing isogenic cardiomyocytes derived from a source isogenic pluripotent stem cells (iPSCs) with isogenic nonmyocytes derived from a source isogenic pluripotent stem cells (iPSCs) for about 1 day to about 10 days (e.g., about 1 to about 2, 3, 4, 5, 6, 7, 8, 9, or 10 days or any value or range therein, e.g., about 1 day to about 5 days) to form a self-assembled 3D cardiac organoid under normoxic conditions; thereby generating the 3D cardiac organoid.

Another aspect of the present invention is directed to a method of making an isogenic 3D cardiac organoid comprising pericytes, the method comprising: culturing isogenic cardiomyocytes source isogenic pluripotent stem cells (iPSCs) with isogenic nonmyocytes source isogenic pluripotent stem cells (iPSCs) for about 1 day to about 10 days (e.g., about 1 to about 2, 3, 4, 5, 6, 7, 8, 9, or 10 days or any value or range therein, e.g., about 1 day to about 5 days) to form a self-assembled 3D cardiac organoid under normoxic conditions, wherein the nonmyocytes comprise pericytes; thereby generating the 3D cardiac organoid comprising pericytes.

A further aspect of the present invention is directed to a method of enhancing function and/or stability of a 3D cardiac organoid, the method comprising: preparing and/or providing cardiomyocytes with nonmyocyte fibroblasts (Fbs), endothelial cells (ECs), pericytes (PCs), and/or mesenchymal cells (MSCs); culturing cardiomyocytes and nonmyocytes in combination with nonmyocyte pericytes (PCs) for about 1 day to about 10 days (e.g., about 1 to about 2, 3, 4, 5, 6, 7, 8, 9, or 10 days or any value or range therein, e.g., about 1 day to about 5 days) to form a self-assembled 3D cardiac organoid under normoxic conditions, wherein the prescence of pericytes enhances organoid function and/or stability (e.g., engraftment in vivo, e.g., structural integrity over time); thereby generating the 3D cardiac organoid comprising pericytes with enhanced organoid function and/or stability.

An additional aspect of the present invention is directed to a method of treating a subject in need thereof, comprising administering to the subject a therapeutically effective amount of an isogenic three-dimensional (3D) cardiac organoid, wherein the isogenic 3D cardiac organoid comprises four different cardiac cell types that comprise cardiomyocytes (CMs) and nonmyocytes, wherein the cardiomyocytes and nonmyocytes are derived from (i.e., source) isogenic pluripotent stem cells (iPSCs) (e.g., an isogenic 3D cardiac organoid as described herein and/or a 3D cardiac organoid generated by a method as described herein). In some embodiments, the therapeutically effective amount of the isogenic three-dimensional (3D) cardiac organoid is administered to the subject via intramyocardial injection.

It is noted that aspects of the invention described with respect to one embodiment, may be incorporated in a different embodiment although not specifically described relative thereto. That is, all embodiments and/or features of any embodiment can be combined in any way and/or combination. Applicant reserves the right to change any originally filed claim and/or file any new claim accordingly, including the right to be able to amend any originally filed claim to depend from and/or incorporate any feature of any other claim or claims although not originally claimed in that manner. These and other objects and/or aspects of the present invention are explained in detail in the specification set forth below. Further features, advantages and details of the present invention will be appreciated by those of ordinary skill in the art from a reading of the figures and the detailed description of the preferred embodiments that follow, such description being merely illustrative of the present invention.

### BRIEF DESCRIPTION OF THE DRAWINGS

FIG. 1 shows primary cell cardiac organoid development. (Panel A) A schematic illustrating organoid preparation by sequential seeding of nonmyocyte supporting cells onto human cardiac spheroids. (Panels B, C) hPSC-CMs organize on the peripheral region of cardiac organoids. (Panel D) Stromal cells are distributed across cardiomyocyte regions and endothelial cells (Panel E) form lumen-like structures (*).

FIG. 2 shows differentiation of 19-9-11 hiPSCs into hPSC-CMs, hPSC-cFbs, and hPSC-ECs. (Panel A) Schematic illustrating the differentiation of hPSCs into hPSC-CMs, hPSC-cFbs, and hPSC-ECs. (Panels C, D, F) Immunofluorescent staining of nuclei (blue) and (Panel B) hPSC-CMs with αSA (green), (Panel D) hPSC-cFbs with vimentin (green), and (Panel F) hPSC-ECs with CD31 (green). (Panels C, E, G) Flow cytometry showing (Panel C) hPSC-CMs stained with cTnT (red), (Panel E) hPSC-cFbs stained with vimentin (red), and (Panel G) hPSC-ECs stained with CD31 (red). Corresponding isotype controls shown in gray.

FIG. 3 shows development of 3- and 4-cell type isogenic hPSC-derived cardiac organoids. (Panel A) (i) Fabrication of 3-cell type cardiac organoids and (ii) representative phase contrast image of 3-cell type organoids on Day 5. (Panel B) (i) Fabrication of 4-cell type cardiac organoids and (ii) representative phase contrast image of 4-cell type organoids on Day 5. (Panel C) Measurement of organoid diameter of 3- and 4-cell type organoids. (Panel D) Contractile capacity of 3- and 4-cell type organoids, measured by percent FAC. (Panels E-J) Immunofluorescent staining of (Panels E-G) 3-cell type organoids and (Panels H-J) 4-cell type organoids. (Panels E-F, H-I) hPSC-CM (a-SA, green) and hPSC-cFb (vimentin, red) organization. (Panels G, J) hPSC-ECs (CD31 , green) and hPSC-cFbs (vimentin, red) in cardiac organoids. ns =no significance.

FIG. 4 shows that incorporation of hPSC-PCs supports vascular development and cell survival in normoxia and hypoxia. (Panels A-B) Immunofluorescent staining of hPSC-ECs (CD31 , green) in (Panel A) 3-and (Panel B) 4-cell type cardiac organoids at Day 5. (*) indicate formation of lumen structures. (Panel C) Quantification of percent area CD31 in 3- and 4-cell type organoids at Day 5. (Panel D) Quantification of the number of lumens per organoid in 3- and 4-cell type organoids at Day 5. (Panels E-F) Immunofluorescent staining of CD31 expression in (Panel E) 3- and (Panel F) 4-cell type organoids after 10 days of culture in (i) normoxia, (ii) 10% oxygen, or (iii) 3% oxygen. (Panels G-I) Corresponding percent area CD31 quantification, comparing (Panel G) 3- vs. 4-cell type organoids at Day 15 in normoxia, (Panel H) 3-cell organoids in normoxia, 10% oxygen, and 3% oxygen, and (Panel I) 4-cell type organoids in normoxia, 10% oxygen, and 3% oxygen. (Panel J) Quantification of TUNEL positive nuclei at Day 15 in normoxia, 10% oxygen, and 3% oxygen culture conditions. ns=no significance, *=p<0.05, **=p<0.01, ***=p<0.005.

FIG. 5 shows enhancement of sarcomere development and expression in 3- and 4-cell type organoids. (Panels A-D) Representative immunofluorescent staining of hPSC-CMs (αSA, green) in (Panel A) hPSC-CM only spheroids, (Panel B) organoids comprised of 70% hPSC-CMs: 30% hPSC-cFbs, (Panel C) 3-cell type organoids, and (Panel D) 4-cell type organoids at Day 5. (Panel E) Quantification of corresponding sarcomere length at Day 5. (Panel F) Quantification of corresponding Z-line width of sarcomeres at Day 5. (Panels G-J) RT-qPCR of hallmark genes related to sarcomere development. ns=no significance, *=p<0.05, **=p<0.01, ***=p<0.005, ****=p<0.001.

FIG. 6 shows enhanced hPSC-CM expression in 4-cell type cardiac organoids after 10 days of hypoxic culture. (Panels A-B) Immunofluorescent staining of hPSC-CMs (αSA, green) in (Panel A) 3- and (Panel B) 4-cell type organoids after 10 days of culture in (i) normoxia, (ii) 10% oxygen, or (iii) 3% oxygen. (Panels C-D) Quantification of percent area αSA in (Panel C) 3-cell type organoids in normoxia, 10% oxygen, and 3% oxygen and (Panel D) 4-cell type organoids in normoxia, 10% oxygen, and 3% oxygen. (Panel E) Comparison of percent area αSA between 3- and 4-cell type organoids cultured for 10 days in normoxia, 10% oxygen, and 3% oxygen. ns=no significance, *=p<0.05, **=p<0.01.

FIG. 7 shows scRNA-seq Characterization of 3- and 4-cell-type cardiac organoids. (Panel A) Annotated UMAP highlighting key populations of organoids, including different hPSC-CM subtypes, hPSC-cFbs, hPSC-ECs, and hPSC-PCs. (Panel B) DimPlots highlighting localization of key annotation markers. (Panels C-F) Percent of total cells in 3- and 4-cell type cardiac organoids of (Panel C) ventricular-like hPSC-CMs, (Panel D) hPSC-cFbs, (Panel E) hPSC-ECs, and (Panel F) hPSC-PCS.

FIG. 8 shows 4-cell type cardiac organoid engraftment and functional recovery of infarcted rat hearts. (Panel A) Schematic showing injury control and organoid injections in I/R-injured athymic rats as well as experimental outline of organoid transplantation. (Panels B-C, E-F) H&E staining of engrafted organoids in (Panels B-C) primary cell organoid-treated rat hearts and (Panels E-F) 4-cell type isogenic organoid-treated rat hearts at Day 28. (Panels D, G) Immunofluorescent staining of TnI (red) and HNA (green) in organoid engraftments from (Panel D) primary cell organoids and (Panel G) 4-cell type organoids at Day 28. (Panels H-J) Representative M-mode echocardiographs at Day 28 for (Panel H) control, (Panel I) primary cell organoid-treated, and (Panel J) 4-cell type organoid-treated I/R-injured rat hearts. (Panel K) FS of rat hearts pre-I/R, post-I/R, 7 days, and 28 days after transplantation. (Panel L) Average percent change in FS at Day 7 and Day 28 post-transplantation compared to post-I/R (from left to right, control, primary cell organoid, 4-cell isogenic organoid). (Panel M) Average percent recovery of FS lost to injury at Day 7 and Day 28. *=p<0.05, **=p<0.01.

FIG. 9 shows attenuation of adverse left ventricular remodeling following 4-cell type cardiac organoid injection in I/R injured rat hearts. (Panel A) Representative picrosirius stains of infarcted rat hearts (dark gray, collagen I/III; light gray, myofibers). (Panel B) Average fibrosis of left ventricle at Day 28 (from left to right, control, primary cell organoid, 4-cell isogenic organoid). (Panel C) Average LV wall thickness at Day 28. (Panel D) Representative images of wheat germ agglutinin (WGA)-stained host cardiomyocytes in border and remote zones of injured hearts at Day 28 (from left to right, control, primary cell organoid, 4-cell isogenic organoid). (Panel E) Quantification of the average cross-sectional area of host cardiomyocytes in the border and remote zones of the host heart. (Panel F) Vessels in the infarct zone at Day 28. (Panel G) Quantification of average infarct vascular density in hearts treated with primary cell or 4-cell isogenic organoids at Day 28 (from left to right, primary cell organoid, 4-cell isogenic organoid). *=p<0.05, **=p<0.01, ***=p<0.005, ****=p<0.001.

FIGS. 10A-10B shows (FIG. 10A) a schematic of an exemplary strategy for preparing pre-vascularized, nanowired human cardiac organoids and (FIG. 10B) staining images of cardiac organoids, showing cardiomyocytes, vascular cells, non-cardiomyocytes, and nanowires.

FIGS. 11A-11D shows (FIG. 11A) a schematic of an exemplary strategy for implanting unwired and nanowired cardiac organoids in ischemia/reperfusion (I/R) injured athymic rats, and graphs of (FIG. 11B) fractional shortening (%) (from left to right, control, unwired, wired), (FIG. 11C) change in fractional shortening post-ischemia/reperfusion (I/R) injury (%), and (FIG. 11D) percent fractional shortening recovery of I/R injury, for control, unwired, and nanowired cardiac organoids.

FIG. 12 is a schematic of an exemplary strategy for establishing hypoimmune organoids. FIG. 13 shows graphs of (Panel A) fractional shortening (%), (Panel B) change in fractional shortening post-ischemia/reperfusion (I/R) injury (%) (from left to right, control, primary cell organoid, wild-type organoid, UDET organoid), and (Panel C) percent fractional shortening recovery of I/R injury, for primary cell organoids, wild-type organoids, and UDET organoids (from left to right, control, primary cell organoid, wild-type organoid, UDET organoid). FIG. 14 shows staining images of 19-9-11 cardiac organoids and UDET cardiac organoids implanted in allogeneic humanized mice.

FIG. 15 shows box plots of cardiac troponin T-positive (cTnT+area) (μm) and average cTnT+area per graft (%) in 19-9-11 cardiac organoids and UDET organoids implanted in allogeneic humanized mice.

### DETAILED DESCRIPTION

The present invention now will be described hereinafter with reference to the accompanying drawings and examples, in which embodiments of the invention are shown. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. Thus, the invention contemplates that in some embodiments of the invention, any feature or combination of features set forth herein can be excluded or omitted. In addition, numerous variations and additions to the various embodiments suggested herein will be apparent to those skilled in the art in light of the instant disclosure, which do not depart from the instant invention. Hence, the following descriptions are intended to illustrate some particular embodiments of the invention, and not to exhaustively specify all permutations, combinations, and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

All publications, patent applications, patents and other references cited herein are incorporated by reference in their entireties for the teachings relevant to the sentence and/or paragraph in which the reference is presented.

Unless the context indicates otherwise, it is specifically intended that the various features of the invention described herein can be used in any combination. Moreover, the present invention also contemplates that in some embodiments of the invention, any feature or combination of features set forth herein can be excluded or omitted. To illustrate, if the specification states that a composition comprises components A, B and C, it is specifically intended that any of A, B or C, or a combination thereof, can be omitted and disclaimed singularly or in any combination.

As used in the description of the invention and the appended claims, the singular forms “a,” “an” and “the” are intended to include the plural forms as well, unless the context clearly indicates otherwise.

Also as used herein, “and/or” refers to and encompasses any and all possible combinations of one or more of the associated listed items, as well as the lack of combinations when interpreted in the alternative (“or”).

Where a range of values is provided, it is understood that each intervening value, to the tenth of the unit of the lower limit unless the context clearly dictates otherwise, between the upper and lower limit of that range and any other stated or intervening value in that stated range, is encompassed within the invention. The upper and lower limits of these smaller ranges may independently be included in the smaller ranges and are also encompassed within the invention. Where the stated range includes one or both of the limits, ranges excluding either or both of those included limits are also included in the invention.

The term “about,” as used herein when referring to a measurable value such as an amount or concentration and the like, is meant to encompass variations of ±10%, ±5%, ±1%, ±0.5%, or even ±0.1% of the specified value as well as the specified value. For example, “about X” where X is the measurable value, is meant to include X as well as variations of ±10%, ±5%, ±1%, ±0.5%, or even ±0.1% of X. A range provided herein for a measurable value may include any other range and/or individual value therein.

As used herein, phrases such as “between X and Y” and “between about X and Y” should be interpreted to include X and Y. As used herein, phrases such as “between about X and Y” mean “between about X and about Y” and phrases such as “from about X to Y” mean “from about X to about Y.”

As used herein, the term “enhance” or “increase,” “increases,” “increased,” “increasing,” and similar terms indicate to an increase in the specified parameter of at least about 1.25-fold, 1.5-fold, 2-fold, 3-fold, 4-fold, 5-fold, 6-fold, 8-fold, 10-fold, twelve-fold, or even fifteen-fold and/or can be expressed in the enhancement and/or increase of a specified parameter of at least about 1%, 5%, 10%, 15%, 25%, 35%, 40%, 50%, 60%, 75%, 80%, 90%, 95%, 100%, 150%, 200%, 300%, 400%, 500% or more.

“Inhibit,” “reduce,” or “mitigate,” or grammatical variations thereof as used herein refers to a decrease or diminishment in the specified parameter of at least about 1, 5, 10, 15%, 25%, 35%, 40%, 50%, 60%, 75%, 80%, 90%, 95% or more. In particular embodiments, the inhibition or reduction results in little or essentially no detectible activity and/or amount of the specified parameter (at most, an insignificant amount, e.g., less than about 10% or even 5%).

An “effective” amount as used herein is an amount that provides a desired effect.

A “therapeutically effective” amount as used herein is an amount that provides some improvement or benefit to the subject. Alternatively stated, a “therapeutically effective” amount is an amount that will provide some alleviation, mitigation, or decrease in at least one clinical symptom in the subject. Those skilled in the art will appreciate that the therapeutic effects need not be complete or curative, as long as some benefit is provided to the subject. Determination of a therapeutically effective amount, as well as other factors related to effective administration of a compound of the present invention to a subject of this invention, including dosage forms, routes of administration, and frequency of dosing, may depend upon the particulars of the condition that is encountered, including the subject and condition being treated or addressed, the severity of the condition in a particular subject, the particular compound being employed, the particular route of administration being employed, the frequency of dosing, and the particular formulation being employed. Determination of a therapeutically effective treatment regimen for a subject of this invention is within the level of ordinary skill in the medical or veterinarian arts. In clinical use, an effective amount may be the amount that is recommended by the U.S. Food and Drug Administration, or an equivalent foreign agency. The amount of active ingredient (e.g., organoid) that can be combined with the carrier materials to produce a single dosage form varies depending upon the subject being treated and the particular mode of administration.

By the terms “treat,” “treating,” or “treatment,” it is intended that the severity of the subject's condition is reduced or at least partially improved or modified and that some alleviation, mitigation or decrease in at least one clinical symptom is achieved.

The terms “prevent,” “preventing,” and “prevention” (and grammatical variations thereof) refer to a delay in the extent or severity of a disease, disorder and/or clinical symptom(s) after onset relative to what would occur in the absence of carrying out the methods of the invention prior to the onset of the disease, disorder and/or clinical symptom(s). The prevention can be complete, e.g., the total absence of the infection, disease, condition and/or clinical symptom(s). The prevention can also be partial, such that the occurrence of the disease, disorder and/or clinical symptom(s) in the subject and/or the severity of onset is less than what would occur in the absence of the present invention. Thus, the terms “prevent,” “preventing,” and “prevention” and like terms are used herein to include imparting any level of prevention or protection which is of some benefit to a subject, such that there is a reduction in the incidence and/or the severity of the disease in a treated subject, regardless of whether the protection or reduction in incidence and/or severity is partial or complete. In some embodiments of the invention, preventing can mean reducing the risk of a immune rejection of a cardiac organoid upon implantation in a subject.

“Pharmaceutically acceptable” as used herein means that the compound or composition is suitable for administration to a subject to achieve the treatments described herein, without unduly deleterious side effects in light of the severity of the disease and necessity of the treatment.

As used herein, the term “comprising”, which is synonymous with “including”, “containing”, and “characterized by”, is inclusive or open-ended and does not exclude additional, unrecited elements and/or method steps. “Comprising” is a term of art that means that the named elements and/or steps are present, but that other elements and/or steps can be added and still fall within the scope of the relevant subject matter.

As used herein, the phrase “consisting of” excludes any element, step, or ingredient not specified in the claim. When the phrase “consists of” appears in a clause of the body of a claim, rather than immediately following the preamble, it limits only the element set forth in that clause; other elements are not excluded from the claim as a whole.

As used herein, the transitional phrase “consisting essentially of” means that the scope of a claim is to be interpreted to encompass the specified materials or steps recited in the claim and those that do not materially affect the basic and novel characteristic(s) of the claimed invention. Thus, the term “consisting essentially of” when used in a claim of this invention is not intended to be interpreted to be equivalent to “comprising.”

“Cells” as used herein are, in general, mammalian cells, such as dog, cat, cow, goat, horse, sheep, mouse, rabbit, rat, etc. cells. In some preferred embodiments the cells are human cells. Suitable cells are known and are commercially available, and/or may be produced in accordance with known techniques. In some embodiments, the cells are harvested from a donor and passaged. In some embodiments, the cells are differentiated from cell lines. In some embodiments, the cells are derived from adult stem cells (bone marrow, peripheral blood, umbilical cord blood, Wharton's jelly in the umbilical cord or from placental tissues), embryonic stem cells, amniotic fluid stem cells, or any other source of stem cells that can be differentiated into the tissue of interest.

“Mammalian” as used herein refers to both human subjects (and cells sources) and non-human subjects (and cell sources or types), such as dog, cat, mouse, monkey, etc. (e.g., for veterinary purposes).

“Extracellular matrix” (or “ECM”) as used herein refers to extracellular molecules secreted by cells that provides structural and biochemical support to the surrounding cells. The ECM is normally composed of an interlocking mesh of fibrous proteins and polysaccharides such as glycosaminoglycans (GAGs). “Extracellular matrix composition” as used herein refers to a composition including ECM proteins. “Extracellular matrix proteins” (or “ECM proteins”) as used herein are known, and include but are not limited to those described in Y. Zhang et al., US Patent Application Publication No. 2013/0288375. Examples of ECM proteins include, but are not limited to, laminin, collagen type I, collagen type IV, fibronectin and elastin.

“Organoid” as used herein refers to an artificial construct created in vitro to mimic or resemble the functionality and/or histological structure of an organ or portion thereof, such as a human heart (“cardiac organoid”). In some embodiments, an organoid is configured to be implanted in a subject (e.g., human) and/or is an implantable construct.

“Media” or “culture media” as used herein refers to an aqueous based solution that is provided for the growth, viability, or storage of cells used in carrying out the present invention. A media or culture media may be natural or artificial. A media or culture media may include a base media and may be supplemented with nutrients (e.g., salts, amino acids, vitamins, trace elements, antioxidants) to promote the desired cellular activity, such as cell viability, growth, proliferation, and/or differentiation of the cells cultured in the media. A “base media,” as used herein, refers to a basal salt nutrient or an aqueous solution of salts and other elements that provide cells with water and certain bulk inorganic ions essential for normal cell metabolism and maintains intra-cellular and/or extra-cellular osmotic balance. In some embodiments, a base media may include at least one carbohydrate as an energy source and/or a buffering system to maintain the medium within the physiological pH range. Examples of commercially available base media may include, but are not limited to, phosphate buffered saline (PBS), Dulbecco's Modified Eagle's Medium (DMEM), Minimal Essential Medium (MEM), Basal Medium Eagle (BME), Roswell Park Memorial Institute Medium (RPMI) 1640, MCDB 131, Click's medium, McCoy's 5A Medium, Medium 199, William's Medium E, insect media such as Grace's medium, Ham's Nutrient mixture F-10 (Ham's F-10), Ham's F-12, α-Minimal Essential Medium (αMEM), Glasgow's Minimal Essential Medium (G-MEM) and Iscove's Modified Dulbecco's Medium. See, e.g., US Patent Application Publication No. US20150175956.

“Hydrogel” as used herein refers to naturally-derived hydrogels and synthetic hydrogels, or a combination thereof.

As used herein, the term “isogenic” refers to genetically identical and/or immunologically compatible. As used herein, genetically identical cells may refer to cells derived from one or more pluripotent and/or progenitor stem cells and/or cell lines, e.g., from one pluripotent and/or progenitor stem cell line and/or from two or more pluripotent and/or progenitor stem cell lines wherein the stem cell lines are themselves isogenic.

In some embodiments, “isogenic” may be used to refer to cells wherein the complete genetics of the cells may not be identical but the cells may still be immunologically compatible, i.e., human leukocyte antigen (HLA)/major histocompatibility complex (MHC)-matched. HLA/MHC matched cells may derive from, for example, allogeneic sources (from the same species but not from the same source; e.g., from a HLA/MHC-matched donor) and/or autologous sources (from the same source; e.g., from the same subject; e.g., from the same patient).

Additional description of the terms as used herein, such as but not limited to “nanowire(s)” and/or “semiconductor nanomaterial(s)”, can be found in U.S. Pat. No. 10,988,735, the disclosures of which are incorporated herein by reference in their entirety.

Additional description of the terms as used herein can also be found in Tan et al. 2023 Sci Adv 9:eadf2898: 1-15; Richards et al. 2017 Biomaterials 142:112-123; Coyle et al. 2020 J tissue Eng Regen Med pages 1-14; Giacomelli et al. 2020 Cell Stem Cell 26:862-879; and Voges et al., 2023 Cell Reports 42(5): 112322, the disclosures of each of which are incorporated herein by reference in their entirety.

The present invention is based, in part, on the development of an isogenic three-dimensional (3D) cardiac organoid, comprising at least four different cardiac cell types that comprise cardiomyocytes (CMs) and nonmyocytes, wherein the cardiomyocytes and nonmyocytes are derived from (i.e., source) isogenic pluripotent stem cells (iPSCs). Advantageously, isogenic 3D cardiac organoids of the present invention may provide a structure and/or function that mimics or resembles the structure and/or function of native cardiac tissue (e.g., native human cardiac tissue). For example, isogenic 3D cardiac organoids of the present invention include cardiomyocytes and nonmyocytes derived from (i.e., source) isogenic pluripotent stem cells (iPSCs) and may support these iPSCs to differentiate so as to mimic or resemble native cardiac tissue (e.g., native human cardiac tissue). In addition, isogenic 3D cardiac organoids of the present invention may comprise cardiomyocytes (CMs) and nonmyocytes (e.g., endothelial cells and/or pericytes) in an amount (e.g., concentration) that mimics the amount (e.g., concentration) of cardiomyocytes and nonmyocytes (e.g., endothelial cells and/or pericytes) found in native cardiac tissue (e.g., native human cardiac tissue). Accordingly, isogenic 3D cardiac organoids of the present invention may be suitable for implantation in a subject (e.g., in the heart of a subject, such as a human subject, for example via intramyocardial injection) and regeneration of cardiac tissue in the subject (e.g., following an ischemia/reperfusion (I/R) injury, myocardial infarction, and/or heart failure). In addition, isogenic 3D cardiac organoids of the present invention, upon implantation in a subject, may reduce or mitigate adverse cardiac (e.g., left ventricular) remodeling in the subject, such as after I/R injury (e.g., myocardial infarction), including reducing or mitigating cardiac fibrosis, left ventricular wall thinning, and/or cardiomyocyte hypertrophy.

Provided according to embodiments of the present invention is an isogenic 3D cardiac organoid comprising four different cardiac cell types. In some embodiments, the four different cardiac cell types comprise cardiomyocytes (CMs) and nonmyocytes, wherein the cardiomyocytes and nonmyocytes are derived from (i.e., source) isogenic pluripotent stem cells (iPSCs).

In some embodiments, the organoid comprises, consists essentially of, or consists of iPSC-CMs (e.g., human iPSC-CMs), iPSC-derived fibroblasts (iPSC-Fbs; e.g., human iPSC-derived cardiac fibroblasts), iPSC-derived endothelial cells (iPSC-ECs; e.g., human iPSC-ECs), and iPSC-derived pericytes (iPSC-PCs; e.g., human iPSC-PCs). In some embodiments, the organoid comprises human induced PSC-CMs (hiPSC-CMs). In some embodiments, the organoid comprises hiPSC-CMs, human adult cardiac fibroblasts (hAcFbs), human umbilical vein endothelial cells (HUVECs), and human adipose derived stem cells (hADSCs). In some embodiments, the organoid does not comprise human adult cardiac fibroblasts (hAcFbs), human umbilical vein endothelial cells (HUVECs), human adipose derived stem cells (hADSCs), or any combination thereof. In some embodiments, an isogenic 3D cardiac organoid of the present invention comprises four different cardiac cell types of that comprise CMs cardiomyocytes and nonmyocytes derived from two or more different donors. In some embodiments, an isogenic 3D cardiac organoid of the present invention comprises four different cardiac cell types that comprise cardiomyocytes and nonmyocytes derived from a single donor.

In some embodiments, cardiomyocytes and/or nonmyocytes present in an isogenic 3D cardiac organoid of the present invention are from a mammal (e.g., a human, a nonhuman primate, a domesticated mammal (e.g., a dog, a cat, a rabbit, a guinea pig, a rat), or a livestock and/or agricultural mammal (e.g., a horse, a bovine, a pig, a goat)). In some embodiments, cardiomyocytes and/or nonmyocytes present in an isogenic 3D cardiac organoid of the present invention are from a human. In some embodiments, cardiomyocytes and/or nonmyocytes present in an isogenic 3D cardiac organoid of the present invention are from a subject (e.g., a patient). “Subject” or “patient” as used herein refers to, in general, a human subject, although aspects of the invention may be implemented with other animal subjects, particularly mammalian subjects (e.g., dogs, cats, horses, goats, sheep) for veterinary and/or research purposes. Subjects may be male or female and of any age, including infant, juvenile, adolescent, adult, and geriatric. In some embodiments, cardiomyocytes and/or nonmyocytes present in an isogenic 3D cardiac organoid of the present invention may be from a subject (e.g., a human) undergoing therapy or in need of therapy for a cardiac disease. In some embodiments, the subject may have a cardiac disease, may have had a myocardial infarction, may have heart failure, and/or may have an ischemia/reperfusion (I/R) injury. In such cases, an isogenic 3D cardiac organoid of the present invention developed from these cells may be used for development of a personalized therapeutic protocol for the subject.

In some embodiments, cardiomyocytes and/or nonmyocytes present in an isogenic 3D cardiac organoid of the present invention are derived from isogenic (MHC/HLA-matched) autologous pluripotent stem cells (PSCs). In some embodiments, cardiomyocytes and/or nonmyocytes present in an isogenic 3D cardiac organoid of the present invention are derived from isogenic (MHC/HLA-matched) allogeneic pluripotent stem cells (PSCs). In some embodiments, deriving the cardiomyocytes and/or nonmyocytes from isogenic (MHC/HLA-matched) autologous or allogenic pluripotent stem cells (PSCs) may reduce and/or prevent immune rejection by a recipient to which an isogenic 3D cardiac organoid of the present invention is implanted and/or administered.

In some embodiments, cardiomyocytes present in an isogenic 3D cardiac organoid of the present invention comprise induced pluripotent stem cell-derived cardiomyocytes (iPSC-CMs), cardiac progenitor cells, primary cardiomyocytes, or any combination thereof.

In some embodiments, nonmyocytes present in an isogenic 3D cardiac organoid of the present invention comprise pericytes (PCs). In some embodiments, nonmyocytes present in an isogenic 3D cardiac organoid of the present invention comprise fibroblasts (Fbs), endothelial cells (ECs), pericytes (PCs) and/or mesenchymal stem cells (MSCs). In some embodiments, an isogenic 3D cardiac organoid of the present invention which comprises pericytes has enhanced endothelial cell and cardiomyocyte survival in hypoxic environments. In some embodiments, nonmyocytes present in an isogenic 3D cardiac organoid of the present invention comprise pericytes (PCs) and the pericytes express PDL1, which may promote immune tolerance in a recipient to which the organoid is implanted and/or administered.

An isogenic 3D cardiac organoid of the present invention may be a hypoimmune organoid. Accordingly, in some embodiments, cardiomyocytes and/or nonmyocytes present in an isogenic 3D cardiac organoid of the present invention comprise a hypoimmune and/or immune evading modification. In some embodiments, source isogenic pluripotent stem cells (iPSCs) from which cardiomyocytes and/or nonmyocytes present in an isogenic 3D cardiac organoid of the present invention are derived comprise a hypoimmune and/or immune evading modification. In some embodiments, the hypoimmune and/or immune evading modification is a beta-2 microglobulin (2M) mutation rendering β2M non-functional. In some embodiments, the hypoimmune and/or immune evading modification comprises transduction with Universal Donor HLA-E trimer (“UDET”). In some embodiments, the hypoimmune and/or immune evading modification comprises a CD47 knock-in modification. In some embodiments, providing cardiomyocytes and/or nonmyocytes comprising a hypoimmune and/or immune evading modification and/or deriving cardiomyocytes and/or nonmyocytes from source isogenic pluripotent stem cells (iPSCs) comprising a hypoimmune and/or immune evading modification may reduce and/or prevent immune rejection by a recipient to which the organoid is implanted and/or administered, optionally without the use of an immunosuppressant.

In some embodiments, an isogenic 3D cardiac organoid of the present invention comprises nanowires. In some embodiments, an isogenic 3D cardiac organoid of the present invention beats spontaneously. “Beats spontaneously” as used herein refers to spontaneous (e.g., involuntary) contraction of the organoid without external stimulation, such as electrical or chemical stimulation. In some embodiments, an isogenic 3D cardiac organoid of the invention may spontaneously beat in a synchronous manner (e.g., as in healthy native cardiac tissue). In some embodiments, when synchrony of beat is measured in a population of isogenic 3D cardiac organoids of the present invention, all of the organoids in the population may beat synchronously. In some embodiments, when synchrony of beat is measured in a population of isogenic 3D cardiac organoids of the present invention, some of the organoids in the population may beat synchronously and others in the population may beat asynchronously.

In some embodiments, cardiomyocytes and nonmyocytes are present in an isogenic 3D cardiac organoid of the present invention in a ratio of about 95:5, 90:10, 85:15, 80:20, 75:25, 70:30, 65:35, 60:40, 55:45, or 50:50 to about 45:55, 40:60, 35:65, 30:70, 25:75, 20:80, 15:85, 10:90, or 5:95 (cardiomyocytes: nonmyocytes). In some embodiments, cardiomyocytes and nonmyocytes are present in an isogenic 3D cardiac organoid of the present invention in a ratio of about 95:5, 90:10, 85:15, 80:20, 75:25, 70:30, 65:35, 60:40, 55:45, 50:50, 45:55, 40:60, 35:65, 30:70, 25:75, 20:80, 15:85, 10:90, or 5:95 (cardiomyocytes: nonmyocytes). In some embodiments, nonmyocytes present in an isogenic 3D cardiac organoid of the present invention comprise fibroblasts in amount of about 15%, 20%, 25%, or 30% to about 35%, 40%, 45%, or 50% based on the total number of nonmyocytes, endothelial cells in an amount of about 25%, 30%, 35%, 40%, 45%, or 50% to about 55%, 60%, 65%, 70%, or 75% based on the total number of nonmyocytes, and pericytes in an amount of about 5%, 10%, or 15% to about 20%, 25%, or 30% based on the total number of nonmyocytes. In some embodiments, nonmyocytes present in an isogenic 3D cardiac organoid of the present invention comprise fibroblasts in amount of about 15%, 20%, 25%, 30%, 35%, 40%, 45%, or 50% based on the total number of nonmyocytes, endothelial cells in an amount of about 25%, 30%, 35%, 40%, 45%, 50%, 55%, 60%, 65%, 70%, or 75% based on the total number of nonmyocytes, and pericytes in an amount of about 5%, 10%, 15%, 20%, 25%, or 30% based on the total number of nonmyocytes. In some embodiments, an isogenic 3D cardiac organoid of the present invention comprises about 70% hPSC-CMs, about 10% hPSC-cFbs, about 15% hPSC-ECs, and about 5% hPSC-PCs.

In some embodiments, cells present in an isogenic 3D cardiac organoid of the present invention are not present in a hydrogel (e.g., a 3D hydrogel or scaffold). In some embodiments, an isogenic 3D cardiac organoid of the present invention is devoid of a hydrogel. In some embodiments, an isogenic 3D cardiac organoid of the present invention is devoid of an exogenously added extracellular matrix composition. In some embodiments, an isogenic 3D cardiac organoid of the present invention self-assembles and, thus, cells present in the organoid deposit an extracellular matrix composition, for example comprising macromolecules (e.g., proteins, polysaccharides, etc.) produced by cells (e.g., fibroblast cells and/or endothelial cells) present in the organoid.

An isogenic 3D cardiac organoid of the present invention can be in any suitable shape. For example, in some embodiments, an isogenic 3D cardiac organoid of the present invention can be in the shape of a spheroid. In some embodiments, an isogenic 3D cardiac organoid of the present invention is porous and contains lumen-like structure(s). In some embodiments, an isogenic 3D cardiac organoid of the present invention (e.g., in the shape of a spheroid) has an average diameter of about 400 microns (μm) or less (e.g., about 400, 350, 300, 250, 200, or 150 μm or less or any value or range therein).

Advantageously, isogenic 3D cardiac organoids of the present invention may be suitable for implantation in a subject (e.g., in the heart of a subject, such as a human subject, for example via intramyocardial injection) and optionally for regeneration of functional cardiac tissue in the subject (e.g., following an ischemia/reperfusion (I/R) injury, myocardial infarction, and/or heart failure). In some embodiments, an isogenic 3D cardiac organoid of the present invention is configured to graft to an infarcted and/or failing heart (e.g., upon implantation into a subject).

An isogenic 3D cardiac organoid of the invention may comprise any one or more of the above described features in any combination thereof.

Provided according to embodiments of the present invention is a method of making an isogenic 3D cardiac organoid (e.g., an isogenic 3D cardiac organoid as described herein), the method comprising: culturing isogenic cardiomyocytes derived from a source isogenic pluripotent stem cells (iPSCs) with isogenic nonmyocytes derived from a source isogenic pluripotent stem cells (iPSCs) for about 1, 2, 3, 4, or 5 day(s) to about 6, 7, 8, 9, or 10 days to form a self-assembled isogenic 3D cardiac organoid under normoxic conditions; thereby generating the isogenic 3D cardiac organoid. In some embodiments, the method comprises culturing the cardiomyocytes with the isogenic nonmyocytes for about 1, 2, or 3 day(s) to about 4 or 5 days.

Also provided according to embodiments of the present invention is a method of making an isogenic 3D cardiac organoid comprising pericytes (e.g., an isogenic 3D cardiac organoid comprising pericytes, as described herein), the method comprising: culturing isogenic cardiomyocytes, which are derived from isogenic pluripotent stem cells (iPSCs), with isogenic nonmyocytes, which are derived from isogenic pluripotent stem cells (iPSCs), for about 1, 2, 3, 4, or 5 day(s) to about 6, 7, 8, 9, or 10 days to form a self-assembled isogenic 3D cardiac organoid under normoxic conditions, wherein the nonmyocytes comprise pericytes; thereby generating the isogenic 3D cardiac organoid comprising pericytes. In some embodiments, the method comprises culturing the cardiomyocytes with the isogenic nonmyocytes comprising pericytes for about 1, 2, or 3 day(s) to about 4 or 5 days.

Provided according to embodiments of the present invention is a method of enhancing function and/or stability of a 3D cardiac organoid, the method comprising: preparing and/or providing cardiomyocytes with nonmyocyte fibroblasts (Fbs), endothelial cells (ECs), pericytes (PCs), and/or mesenchymal cells (MSCs); culturing the cardiomyocytes and nonmyocytes, which includes nonmyocyte pericytes (PCs), for about 1, 2, 3, 4, or 5 day(s) to about 6, 7, 8, 9, or 10 days to form a self-assembled 3D cardiac organoid under normoxic conditions, wherein the presence of pericytes in the organoid enhances organoid function and/or stability (e.g., engraftment in vivo, e.g., structural integrity over time); thereby generating the 3D cardiac organoid comprising pericytes with enhanced organoid function and/or stability. In some embodiments, the method comprises culturing the cardiomyocytes and the nonmyocytes in combination with the pericytes for about 1, 2, or 3 day(s) to about 4 or 5 days. In some embodiments, the method enhances the function and/or stability of the 3D cardiac organoid by enhancing and/or improving engraftment in vivo (e.g., a 3D cardiac organoid prepared according to the method provides improved cell viability, cell maturation, functionality, and/or vascularization of the organoid following implantation in a subject). In some embodiments, the method enhances the function and/or stability of the 3D cardiac organoid by enhancing and/or improving structural integrity of the organoid over time.

In some embodiments, a method of making an isogenic 3D cardiac organoid of the present invention comprises culturing cardiomyocytes (e.g., iPSC-CMs) with nonmyocytes (e.g., Fbs, endothelial cells, pericytes, and/or mesenchymal stem cells, such as iPSC-derived endothelial cells, pericytes, and/or mesenchymal stem cells) at a total concentration of about 1×10⁵ cells/mL to about 1×10⁷ cells/mL (e.g., about 1×10⁵, 2×10⁵, 3×10⁵, 4×10⁵, 5×10⁵, 5×10⁵ 6×10⁵, 7×10⁵, 8×10⁵, 9×10⁵, 1×b 10⁶, 2×10⁶, 3×10⁶, 4×10⁶, 5×10⁶, 5×10⁶ 6×10⁶, 7×10⁶, 8×10⁶, 9×10⁶, 1×10⁷ cells/mL, or any value or range therein).

In some embodiments, the cardiomyocytes may be cultured with the nonmyocytes in a cell suspension in microwells composed of non-fouling materials. The cell suspension may comprise one or more culture media suitable for culturing cardiomyocytes and/or nonmyocytes. Culture media for culturing cardiomyocytes and/or nonmyocytes are well known in the art. The type of culture media in a cell suspension can vary. For example, a cell suspension may comprise a larger amount of cardiomyocyte cell culture media when the amount of cardiomyocytes being cultured is greater than the amount of nonmyocytes. In another example, the cell suspension may comprise a larger amount of nonmyocyte cell culture media when the amount of nonmyocytes being cultured is greater than the amount of cardiomyocytes being cultured in the cell suspension. Thus, the amounts of all the specific media may be ratiometric reflecting the cell ratio of the organoid.

The micro-wells employed in the inventive method can be any micro-wells comprising non-fouling materials known in the art that are suitable for microtissue fabrication. In some embodiments, the non-fouling materials comprise agarose or non-adhesive self-assembly plates, such as the InSphero Gravity TRAP ultra-low attachment plate. The non-fouling materials may comprise any suitable material, such as, for example, agarose gel, polyethylene glycol, alginate, hyaluronic acid, polyacryylic acid, polyacrylic amide, polyvinyl alcohol, polyhydroxyethyl methacrylate, methacrylated dextrans, poly(N-isopropylacrylamide), and any combination thereof. In some embodiments, the non-fouling materials are devoid of a hydrogel.

In some embodiments, the cardiomyocytes are cultured with the nonmyocytes thereby forming a self-assembled 3D cardiac organoid under normoxic conditions, wherein normoxic conditions comprise a partial pressure of oxygen in the gas phase of about 16% to about 20.9% of the total barometric pressure (or at least about 16%, about 17%, about 18%, about 19%, or at least about 20% of the total barometric pressure).

A method of making a 3D cardiac organoid of the present invention may further comprise providing nanowires to a culture comprising cardiomyocytes and nonmyocytes (e.g., comprising pericytes), thereby generating a nanowired 3D cardiac organoid (e.g., a nanowired isogenic 3D cardiac organoid as described herein).

In some embodiments, a method of making an isogenic 3D cardiac organoid of the present invention comprises culturing cardiomyocytes and nonmyocytes in the presence of a therapeutic agent. In some embodiments, the therapeutic agent is selected from norepinephrine, angiotensin II, TNF-alpha, interfering RNAs, microRNAs, matrix metalloproteases, growth factors, and any combination thereof. The amount of the therapeutic agent can vary. For example, in some embodiments, the amount of the therapeutic agent may range from about 0.01 μM to about 10 μM, about 1μM to about 8 μM, or from about 3 μM to about 5 μM (e.g., about 1 μM, about 2 μM, about 3 μM, about 4 μM, about 5 μM, about 6 μM, about 7 μM, about 8 μM, about 9 μM, or from about 10 μM, or any range or value therein).

Provided according to the present invention is a method of treating a subject in need thereof, comprising administering to the subject a therapeutically effective amount of an isogenic three-dimensional (3D) cardiac organoid, wherein the isogenic 3D cardiac organoid comprises four different cardiac cell types that comprise cardiomyocytes (CMs) and nonmyocytes, wherein the cardiomyocytes and nonmyocytes are derived from (i.e., source) isogenic pluripotent stem cells (iPSCs). In some embodiments, a method of the present invention comprises administering to a subject a therapeutically effective amount of an isogenic 3D cardiac organoid as described herein and/or a 3D cardiac organoid generated by a method as described herein.

In some embodiments, an isogenic 3D cardiac organoid administered to a subject according to a method of the present invention has enhanced and/or improved engraftment in the subject (e.g., the organoid has improved cell viability, cell maturation, functionality, and/or vascularization of the organoid following administration to a subject), as compared to a control (e.g., as compared to engraftment of a non-isogenic 3D cardiac organoid and/or engraftment of a 3D cardiac organoid devoid of pericytes and/or engraftment of dissociated PSC-CMs).

In some embodiments, a method of the present invention comprises administering to a subject a therapeutically effective amount of an isogenic three-dimensional (3D) cardiac organoid, wherein the therapeutically effective amount of the isogenic 3D cardiac organoid is administered to the subject by implanting the therapeutically effective amount of the isogenic 3D cardiac organoid in the subject (e.g., in the heart of the subject). In some embodiments, the therapeutically effective amount of the isogenic 3D cardiac organoid is administered to the subject during a surgical procedure, such as an open-heart surgery. In some embodiments, the therapeutically effective amount of the isogenic 3D cardiac organoid is administered to the subject via intramyocardial injection. In some embodiments, the subject has a cardiac disease. In some embodiments, the subject is undergoing therapy or in need of therapy for a cardiac disease. In some embodiments, the subject has had a myocardial infarction and/or has an ischemia/reperfusion injury and/or heart failure.

In some embodiments, an organoid and/or method of the present invention may reduce or mitigate adverse cardiac remodeling such as after I/R injury (e.g., myocardial infarction). In some embodiments, an organoid and/or method of the present invention may reduce or mitigate adverse left ventricular (LV) remodeling after I/R injury. In some embodiments, an organoid and/or method of the present invention may reduce or mitigate cardiac fibrosis, LV wall thinning, and/or cardiomyocyte hypertrophy. In some embodiments, an organoid and/or method of the present invention may increase infiltration of host vasculature to an infarct region.

For example, in some embodiments, an organoid and/or method of the present invention may provide less than about 20% LV cardiac fibrosis after I/R injury. In some embodiments, an organoid and/or method of the present invention may reduce LV cardiac fibrosis by at least about 30%, optionally by at least about 50%, as compared to a control organoid (e.g., a non-isogenic 3D cardiac organoid and/or a 3D cardiac organoid devoid of pericytes and/or dissociated PSC-CMs) and/or a control method (e.g., a method using a non-isogenic 3D cardiac organoid and/or a 3D cardiac organoid devoid of pericytes and/or dissociated PSC-CMs). In some embodiments, an organoid and/or method of the present invention may provide an LV infarct wall thickness that is at least about 50% to about 100% greater than the LV infarct wall thickness provided by a control organoid (e.g., a non-isogenic 3D cardiac organoid and/or a 3D cardiac organoid devoid of pericytes and/or dissociated PSC-CMs) and/or a control method (e.g., a method using a non-isogenic 3D cardiac organoid and/or a 3D cardiac organoid devoid of pericytes and/or dissociated PSC-CMs). In some embodiments, an organoid and/or method of the present invention may provide an average number of host cardiomyocytes in a cross-sectional area (μm²) of a remote and/or border zone surrounding an infarct region that is reduced by at least about 50% as compared to the average number of host cardiomyocytes in a cross-sectional area (μm²) of a remote and/or border zone provided by a control organoid (e.g., a non-isogenic 3D cardiac organoid and/or a 3D cardiac organoid devoid of pericytes and/or dissociated PSC-CMs) and/or a control method (e.g., a method using a non-isogenic 3D cardiac organoid and/or a 3D cardiac organoid devoid of pericytes and/or dissociated PSC-CMs).

In some embodiments, an organoid and/or method of the present invention provides an infiltration of host vasculature to an infarct region that is at least about 100% greater, optionally at least about 150% greater, than the infiltration of host vasculature to an infarct region provided by a control organoid (e.g., a non-isogenic 3D cardiac organoid and/or a 3D cardiac organoid devoid of pericytes and/or dissociated PSC-CMs) and/or a control method (e.g., a method using a non-isogenic 3D cardiac organoid and/or a 3D cardiac organoid devoid of pericytes and/or dissociated PSC-CMs).

Having described the present invention, the same will be explained in greater detail in the following examples, which are included herein for illustration purposes only, and which are not intended to be limiting to the invention.

### EXAMPLES

### Example 1

### Enhancing the Therapeutic Potential of Isogenic hPSC-Derived Cardiac Organoids via the Incorporation of hPSC-Derived Pericytes

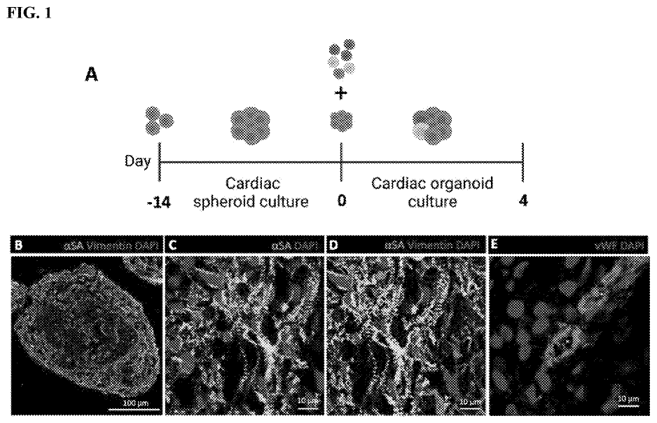

Previous research in our group has focused on the development and therapeutic efficacy of prevascularized human cardiac organoids for heart regeneration^{1,24,25} We explored the biomimetic development of human cardiac organoids by sequentially seeding human adult cardiac fibroblasts (hAcFbs), human umbilical vein endothelial cells (HUVECs), and human adipose derived stem cells (hADSCs) onto pre-formed hPSC cardiac spheroids (100% hPSC-CMs), referred to as “primary cell cardiac organoids” for the remainder of this work (FIG. 1). Upon injection of primary cell organoids into I/R injured rat hearts, we observed significant improvement in the contractile function of I/R injured hearts at Day 28 when compared to control (I/R injured rats without organoid injections)¹. Previous studies reported ~8% Fractional Shortening (FS) improvement after transplantation of 1×10⁷ hPSC-CMs/rat with an identical rat I/R injury model^{26,27}. Remarkably, we achieved ~9% improvement in FS with only 10% of cells (1×10⁶ total cells/rat, 5×10⁵ hPSC-CMs/rat) with primary cell cardiac organoid transplantation.

Despite our progress, the use of primary somatic cells in the cardiac organoids has limited their translational potential because these cells must be human leukocyte antigen (HLA)-matched to the recipient; otherwise, the recipient's immune response will result in the rejection of donor cells²⁸. Isogenic cardiac organoids have the potential to eliminate the threat of immune rejection due to the use of one cell line in their composition. hPSC-derived cardiomyocyte and nonmyocyte populations could come from either an autologous, patient-derived source or an allogenic, HLA-matched source to facilitate successful transplantation. Therefore, to make our cardiac organoids more clinically relevant, we sought to develop an isogenic cardiac organoid model. We began by developing cardiac organoids, termed “3-cell type cardiac organoid” for the remainder of this work, composed of hPSC-CMs, hPSC-derived cardiac fibroblasts (hPSC-cFbs), and hPSC-derived endothelial cells (hPSC-ECs) differentiated from a common hPSC line (19-9-11 hiPSCs).

As prevascularization is a promising strategy in cardiac tissue engineering to improve cell survival following transplantation into infarcted tissue³¹, we hypothesized that introducing hPSC-derived pericytes (hPSC-PCs) derived from the 19-9-11 hiPSC into our 3-cell type cardiac organoid would enhance the vascular network and overall organization and cell survival within our organoids. Here, we demonstrate that isogenic cardiac organoids containing hPSC-PCs, referred to as “4-cell type cardiac organoids” for the remainder of this work, exhibit enhanced development in normal conditions and survival in hypoxic conditions. Furthermore, we show that 4-cell type cardiac organoids facilitate functional recovery in a rat model of ischemia/reperfusion (I/R) injury, comparable to our previous studies involving primary cell cardiac organoids¹.

### Materials and Methods

hPSC culture. hPSCs were cultured as previously described in 32. Briefly, tissue culture plates were coated with hESC-certified Matrigel (Corning Inc., Corning, NY, USA) for >1 h at 37° C., 5% CO₂ according to the manufacturer's recommended dilution factor. hiPSCs from the 19-9-11 cell line, 19-9-11 (WiCell, Madison, WI, USA), were thawed in a 37° C. warm bath and resuspended in mTeSR1 (StemCell Technologies, Vancouver, Canada) and centrifuged at 200×g for 5 minutes at room temperature. Cells were subsequently resuspended in mTeSR 1 with 5 μM Y-27632 (Tocris Bioscience, Bristol, UK) and seeded onto Matrigel-coated well plates. hiPSCs were cultured at 37° C., 5% CO₂ and mTeSR1 media was exchanged daily within 27 h. hiPSCs were passaged once 85-90% confluent using Versene (Thermo Fisher Scientific, Waltham, MA, USA) at 37° C., 5% CO₂ for 5 minutes. Versene was aspirated and hiPSCs were gently flushed off wells with mTeSR1. Collected cells were replated at 1:6 on Matrigel-coated well plates.

hPSC-CM differentiation and culture. hPSC-CM differentiation was performed as described previously in³². The differentiation protocol was based on previously established methods, according to the GiWi protocol 33 to establish cardiac progenitor cells. In brief, 90% confluent 19-9-11 hiPSCs were singularized following incubation in Accutase for 6 minutes at 37° C., 5% CO₂ and seeded onto Matrigel-coated 24-well plates at 600,000 cells/cm² in mTeSR1 with 5 μM Y-27632 on Day −2. Media was exchanged with mTeSR1 24 h later (Day −1). On Day 0, cells were treated with 6-8 μM CHIR 99021 (Selleck Chemicals, Houston, TX, USA) in RPMI 1640 (Thermo Fisher Scientific) supplemented with 2% B 27 Minus Insulin (Thermo Fisher Scientific, Waltham, MA) (RPMI/B27−). Precisely 24 h later (Day 1), media was replaced with fresh RPMI/B27−. On Day 3, cells were treated with RPMI/B27− supplemented with IWP2 (Sigma-Aldrich). Precisely 48 h later (Day 5), media was exchanged with fresh RPMI/B27−. By Day 6, cells were considered to be cardiac progenitor cells. On Day 7, media was replaced with RPMI 1640 supplemented with B27 with Insulin (Thermo Fisher Scientific) (RPMI/B27+). Media was subsequently replaced every other day. On Day 10, cells were purified via lactate purification 34 for 48 h. Lactate purification media consisted of glucose-free DMEM (Thermo Fisher Scientific) supplemented with 3 mM Na-1-lactate (BeanTown Chemical, Hudson, NH, USA). On Day 12, media was replaced with RPMI/B27+ for 2 days of recovery from purification. On Day 14, purified hPSC-CMs were prepared for cryopreservation via dissociation using Accutase at 37° C., 5% CO₂ for 40 minutes. Accutase was quenched 1:1 with RPMI/B27+ and centrifuged at 200×g for 5 minutes at room temperature. hPSC-CMs were resuspended in RPMI/B27+ supplemented with 30% Defined FBS (Cytiva Life Sciences, Marlborough, MA, USA), 10% DMSO (Sigma-Aldrich, St. Louis, MO, USA), and 5 μM Y-27632. hPSC-CMs were frozen overnight at −80° C. in a Nalgene Mr. Frosty Freezing Container (Thermo Fisher Scientific) and transferred to liquid nitrogen for long-term storage. Thawing of cryopreserved hPSC-CMs consisted of seeding cells at 15,000 cells/cm² on Matrigel-coated well plates in RPMI/B27+ supplemented with 10% Defined FBS and 5 μM Y-27632. Expansion of hPSC-CM^{35,36} began 24 h after thaw via replacement of media with RPMI/B27+ supplemented with 3 μM CHIR99021. Media was exchanged with RPMI/B27+ supplemented with 3 μM CHIR99021 every other day for cardiomyocyte expansion.

hPSC-cFb differentiation and culture. hPSC-cFb differentiation was performed as described previously in 32. In summary, 19-9-11 hiPSCs were differentiated based on the GiWi protocol to derive cardiac progenitors33, followed by differentiation into hPSC-derived epicardial cells^{37,38}, and finally into hPSC-cFbs. Briefly, Day 6 cardiac progenitor cells (see “hPSC-CM differentiation” above) were differentiated following seeding 19-9-11 hiPSCs on Day−2 at 550,000 cells/cm² into Matrigel-coated 24-well plates and treated with 8-9 μM CHIR99021 on Day 0. For hPSC-derived epicardial cells, Day 6 cardiac progenitor cells were dissociated using Accutase at 37° C., 5% CO₂ for 10 minutes and replated at 50,000 cells/cm² in LaSR basal media, consisting of Advanced DMEM/F12 (Thermo Fisher Scientific), 10 μg/mL l-Ascorbic Acid (Sigma-Aldrich), 1X GlutaMAX (Thermo Fisher Scientific), and 5 μM Y-27632. On Day 7, cells were treated for 48 h with 8 μM CHIR99021 in LaSR basal media. After 24 h (Day 8), cells were treated with fresh LaSR basal with 8 μM CHIR99021 . After 48-h treatment, media was exchanged with LaSR basal media daily until Day 12. ON Day 12, cells were considered to be differentiated into hPSC-epicardial cells and passaged 1:6 and/or cryopreserved in LaSR basal supplemented with 0.5 μM A83-01 (Abcam, Cambridge, UK), 10% FBS (Sigma-Aldrich), and 10% DMSO (Sigma-Aldrich). hPSC-epicardial cells were thawed in LaSR basal with 10% FBS, 0.5 MM A83-01 and 5 μM Y-27632 on Matrigel-coated plates. For maintenance of hPSC-epicardial cells, media (LaSR basal with 0.5 μM A83-01) was exchanged daily. For passaging, hPSC-epicardial cells were passaged at 1:6 once 90% confluent using TrypLE at 37° C.5% CO₂ for 10 minutes and replated in LaSR basal with 0.5 μM A 83-01 and 5 μM Y-27632 onto Matrigel-coated 6-well plates. Cells were passaged 2-3 times before beginning hPSC-cFb differentiation. hPSC-cFb differentiation began once hPSC-epicardial cells reached 100% confluency on Matrigel-coated plates. The hPSC-epicardial cells were passaged 1:1 onto porcine heart extracellular matrix (HEM)-coated plates (Day−1). HEM-coated plates were prepared as described in³². In brief, HEM solution at 10 mg/mL (preparation described in 39) was diluted in 0.02 M acetic acid to 20 μg/mL and added to well plates. Plates were incubated for >16 h at 37° C., 5% CO₂. Prior to seeding cells, HEM solution was aspirated, and wells were rinsed with 1X DPBS (Thermo Fisher Scientific). hPSC-cFb differentiation began on Day 0 with 10 days of treatment with LaSR basal+10 ng/mL BFGF (Invitrogen, Waltham, MA, USA). Media was changed daily. On Day 10, cells were considered to be hPSC-cFb and passaged onto HEM-coated well plates in FibroGRO Complete Media (Millipore Sigma, Burlington, MA, USA) with 2% FBS and 1X GlutaMAX. Cells were maintained in FibroGRO and passaged 1:6 using TrypLE when they reached 80-90% confluency.

hPSC-EC differentiation and culture. hPSC-EC differentiation was performed as described previously in³². hPSC-EC differentiation was based on previous reports⁴⁰⁻⁴³. Briefly, 80-90% confluent 19-9-11 hPSCs were dissociated with Accutase for 6 min at 37° C., 5% CO₂, quenched with equal parts mTeSR1, and centrifuged at room temperature, 200×g for 5 minutes. Cells were resuspended in mTeSR1 with 5 MM Y-27632 and seeded at 125,000 cells/cm² (Day −2). Media was replaced with mTeSR1 after 24 h (D-1). On Day 0, cells were treated with 6 μM CHIR99021 in LaSR basal for precisely 48 h, replacing with fresh LaSR basal with 6 μM CHIR99021 after 24 h. On Day 2, media was changed to LaSR basal and replaced daily. On Day 5, cells were dissociated with Accutase for 10 min at 37° C., 5% CO₂, quenched with LaSR Basal, and purified using CD34+ Microbeads (Miltenyi Biotec) following the manufacturer's instructions. Purified CD34+ cells were seeded at 10,000/cm² onto collagen I-coated wells (50 μg/mL)in EGM2 (PromoCell, Heidelberg, DEU) supplemented with 0.5 μM A83-01 and 10 μM Y-27632. Media was replaced 1 day after with EGM2 supplemented with 0.5 μM A83-01 (Day 6) and media was further exchanged every other day. On Day 10, cells were purified by treatment with Versene for 5 min to remove non-endothelial cells and rinsed with 1X DPBS. Cells were subsequently replated 1:3 onto fresh collagen I-coated plates after dissociation with TrypLE for 15 min at 37° C., 5% CO₂ and maintained in EGM2 supplemented with 0.5 μM A83-01.

Flow cytometry. Cells were prepared for flow cytometry via dissociation with 1X TrypLE Select (Thermo Fisher Scientific) for 10 min. For intracellular stains, cells were centrifuged at 200×g for 5 min and resuspended in cold 1% Paraformaldehyde for fixation (20 min, 4° C.). Cells were diluted 1:10 with 1X PBS and centrifuged at 200×g for 5 min. Cells were then rinsed once with Intracellular Flow Buffer (1X DPBS, 1% Bovine Serum Albumin (Thermo Fisher Scientific) and 0.1% Triton X-100 (Sigma-Aldrich)) and stored at 4° C. until stained. Fixed cells were centrifuged (200×g, 5 min, room temperature) and resuspended in Intracellular Flow Buffer with primary antibodies. Primary antibodies used for intracellular stains: Rabbit anti-Vimentin (1:200) (Abcam) and Rabbit anti-a-cardiac troponin T (1:200) (Abcam). An unstained control group was incubated in only Intracellular Flow Buffer. Cells were incubated with primary antibody or Intracellular Flow Buffer overnight at 4° C. Post-incubation, all groups were rinsed with Intracellular Flow Buffer, centrifuged (200×g, 5 min, room temperature) and resuspended in Intracellular Flow Buffer with Goat anti-Rabbit Alexa Fluor 647 secondary antibody (Abcam) and incubated for 1 h at room temperature, protected from light. Stained cells were rinsed twice with Flow Buffer and resuspended in 300 μL Flow Buffer for flow cytometry analysis. For cell surface markers, cells were centrifuged at 200×g for 5 min and resuspended in Flow Buffer (1X DPBS, 0.5% Bovine Serum Albumin, 2 mM EDTA) with pre-conjugated antibodies. Antibodies used for cell surface markers: FITC anti-CD31 (1:50) (Biolegend, San Diego, CA, USA), PE anti-NG2 (1:50) (R&D Systems, Minneapolis, MN, USA), FITC anti-CD13 (1:50) (Thermo Fisher Scientific), FITC anti-CD34 (1:50) (Biolegend), and their corresponding isotype controls. Cells were incubated with antibody for 30 minutes at 4°C. Post-incubation, all groups with rinsed with Flow Buffer and centrifuged (200×g, 5 min, room temperature). Stained cells were resuspended in 300 μL Flow Buffer for flow cytometry analysis. Flow cytometry was performed using a MACSQuant Analyzer 10 (Miltenyi Biotec) and analysis performed using FlowJo (FlowJo, Ashland, OR, USA).

2D fluorescent imaging. Cells were seeded and cultured under normal conditions in 24-well plates containing sterile glass coverslips. Once at an appropriate confluency for staining (70-100% confluent), wells containing coverslips were treated with 4% Paraformaldehyde (20 min, room temperature) for fixation. Wells were rinsed with 1X DPBS+Ca²⁺+Mg²⁺ (Thermo Fisher Scientific) twice and stored in 1X DPBS+Ca²⁺+Mg²⁺ at 4° C. until ready for staining. All subsequent steps were performed on coverslips in the well plates. Antigen blocking was performed using 10% Goat Serum (Millipore Sigma) in PBST-0.01(1X DPBS+Ca²⁺+Mg²⁺ supplemented with 0.01% Triton-X) (Sigma-Aldrich) (1 h, room temperature) and then rinsed twice with PBST-0.01 containing Ca²⁺ and Mg²⁺. Cells were then stained with primary antibodies in PBST-0.01 for >16 h at 4° C. Primary antibodies used: Rabbit anti-Vimentin (1:200) (Abcam), Mouse anti-CD31 (1:50) (BD Pharmingen, San Diego, CA, USA), or Rabbit anti-α-sarcomeric actinin (1:200) (Abcloncal, Wuhan, Hubei, CN). Cells were rinsed twice with PBST-0.01 and stained with secondary antibodies for 1 h at room temperature, protected from light. Secondary antibodies used: Goat anti-Rabbit Alexa Fluor 647 (Abcam) (1:200) or Goat anti-Mouse Alexa Fluor 647 (Abcam) (1:200). Stained cells were rinsed once with PBST-0.01 and then stained with NucBlue nuclei stain (Thermo Fisher Scientific) diluted in PBST-0.01 according to the manufacturer's instruction (20 min, room temperature, protected from light). Cells were rinsed twice with PBST-0.01 and then coverslips were removed from well plates. For mounting, coverslips were inverted onto microscope slides and mounted using Fluoroshield (Sigma-Aldrich). Images were taken using a BZ-X All-in-One Fluorescence Microscope (Keyence, Osaka, JP).

hPSC cardiac organoid fabrication. hPSC cardiac organoids were fabricated based on previously published methods^{1,32}. hPSC cardiac organoids were fabricated in a sequential seeding method (i.e., hPSC-CM spheroids (hPSC-CM only microtissues) were fabricated first, followed by seeding with supporting cell types to create hPSC cardiac organoids). To fabricate hPSC-CM spheroids, hPSC-CMs were expanded to super-confluency in RPMI/B27+ with 3 μM CHIR99021 and allowed to recover in RPMI/B27+ for 24 h. hPSC-CM spheroids were fabricated on Day −14 (14 days prior to adding supporting cells). Agarose molds containing 35 microwells for organoid self-assembly were fabricated with 2% agarose, 24-h prior to seeding. Agarose molds were maintained in RMPI/B27+ to prevent drying. hPSC-CMs were dissociated using TrypLE for 40 minutes at 37° C., 5% CO₂. hPSC-CMs were quenched and collected in RPMI/B27+, centrifuged (200×g, 5 min, room temperature), and resuspended to 4×10⁶ cells/mL in RPMI/B27+. For organoids for in vitro studies, 147,000 hPSC-CMs were plated into agarose molds (4,200 hPSC-CMs/microwell). For hPSC-CM only spheroids, 210,000 hPSC-CMs were plated into agarose molds (6,000 hPSC-CMs/microwell). For injection organoids, 70,000 hPSC-CMs were plated into agarose molds (2,000 hPSC-CMs/microwell). hPSC-CMs were permitted to settle in microwells for 20 min before adding RPMI/B27+ to each well. After 14 days of hPSC-CM spheroid culture with media changes every other day, supporting cells were added (Day 0). All cell types were dissociated using TrypLE (37° C., 5% CO₂): 30 min for hPSC-ECs, 15 min for hPSC-cFbs, and 15 min for hPSC-PCs. Cells were quenched and collected in their cell-type specific maintenance media, centrifuged (200×g, 5 min, room temperature), and resuspended to 4×10⁶ cells/mL in organoid media. Organoid media consisted of 48% DMEM/F12 (Thermo Fisher Scientific), 5.4% Defined FBS, 0.54% Non-essential amino acids (Thermo Fisher Scientific), 30% Fibroblast Growth Media-3 (PromoCell), and 16% EGM2. Media was carefully aspirated from spheroid wells and supporting cell mixtures were prepared to plate onto hPSC-CM spheroids with the final cell ratios: 70% hPSC-CM: 15% hPSC-cFb: 15% hPSC-EC for 3-cell type hPSC organoids and 70% hPSC-CM: 10% hPSC-cFb: 15% hPSC-EC: 5% hPSC-PC for 4-cell type hPSC organoids. Cells were permitted to settle into the microwells of the agarose molds for 20 min then filled with organoid media. Organoids were maintained at 37° C., 5% CO₂, and media was exchanged every other day.

hPSC-PC differentiation and culture. hPSC-PC differentiation was based on the mesodermal pericyte induction protocol established by Faal et al.⁴⁴. In brief, 90% confluent 19-9-11 hiPSCs were singularized following incubation in Accutase for 6 minutes at 37° C., 5% CO₂ and seeded onto Matrigel-coated 24-well plates at 200,000 cells/cm² in mTeSR1 with 5 μM Y-27632 on Day −1. On Day 0, cells media was changed to Mesoderm Induction Media (MIM) (StemCell Technologies). MIM was replaced daily for 5 days. On Day 6, cells were replated onto 0.1% gelatin-coated plates in pericyte medium (ScienCell, Carlsbad, CA, USA). Pericyte media was replaced every other day until Day 10. On Day 10, cells were passaged at a concentration of 25,000 cells/cm² and maintained in pericyte medium.

Video analysis of beating organoids. As described in previous reports^{1,32,45}, videos of spontaneously beating organoids were recorded using a Carl Zeiss Axiovert A1 Inverted Microscope and Zen 2011 software (Zeiss) with a frame rate of 14 frames per second. To measure changes in area during contraction and relaxation, videos were converted to a sequence of TIFF format pictures with ImageJ (National Institute of Health). A threshold was applied to high contrast videos of contracting organoids and the area of the organoids was subsequently measured for each frame of the video. The changes in area during contraction and relaxation were measured as a percent change in fractional area change amplitude.

Immunofluorescent staining of in vitro organoids. Organoid immunofluorescent staining was performed as previously described^{1,32}. In brief, one well of organoids was embedded by flash-freezing in Optimal Cutting Temperature Compound (OCT) (Thermo Fisher Scientific) in Tissue-Tek plastic cryomolds (Sakura Finetek, Torrance, CA, USA), and immediately frozen to −80° C. Embedded blocks were sectioned at 8 μm per section using a Cryostat Microtome (Leica Biosystems, Wetzlar, DEU) and mounted on glass slides (Fisher Scientific, Pittsburgh, PA, USA). Mounted sections were stored at −20° C. Sections were then fixed with 100% cold Acetone (12 min) and allowed to dry. Sections were blocked with 10% Goat Serum in 1X DPBS with 0.01% Triton X-100 (PBST-0.01) (1 h, room temperature). Sections were then stained with primary antibodies diluted in PBST-0.01 for >16 h at 4° C. Primary antibodies used: Rabbit anti-α-sarcomeric actinin (αSA) (Abclonal) (1:200), Mouse anti-Vimentin (Abcam) (1:200) or Mouse anti-CD31 (BD Pharmingen) (1:50). Sections were then rinsed for 5 min with PBST-0.01 and then stained with secondary antibodies for 1 h at room temperature, protected from light. Secondary antibodies used: Goat anti-Rabbit Alexa Fluor 647 (Abcam) (1:200), Goat anti-Mouse Alexa Fluor 488 (Abcam) (1:200). Sections were subsequently rinsed for 5 min with PBST-0.01 and then stained with NucBlue for 20 min at room temperature, protected from light. Sections were then rinsed twice for 5 min with PBST-0.01, and a coverslip was mounted using Fluoroshield.

Immunofluorescent image quantification and analysis. Stained organoid sections were imaged using Leica SP5 Confocal microscope (Leica Biosystems). For measuring area of fluorescence of CD31 and αSA, image threshold was performed on 8-bit images of each channel to measure the total area of fluorescent channel. The area of CD31 and αSA was normalized to the area of individual organoids. Quantification of the number of lumens per organoid was accomplished by manually identifying lumen structures (via CD31+ staining) in each organoid cross-section. The average sarcomere length was defined as spacing between αSA striations from black and white renderings of 40× magnification confocal organoid images. Using ImageJ, fluorescence profiles along lines passing perpendicular through 3 different striated regions containing at least 6 consecutive sarcomere structures, were measured, and divided by the total number of sarcomeres. Z-line width was also measured on αSA-stained images by measuring the width of striations from the same regions described above.

Hypoxia treatment and TUNEL assay. On Day 5 of culture, cardiac organoids receiving hypoxic treatment were placed in a hypoxia chamber (ProOX Model 110, BioSpherix, Parish, NY) set at either 10% O₂ or 3% O₂ for 10 days. On Day 15, hypoxia-cultured organoids were flash-frozen in Tissue-Tek OCT compound and cryo-sectioned. A Roche In Situ Cell Death Detection Kit (Sigma Aldrich) Sections were fixed in 4% paraformaldehyde solution for 15 min at room temperature then washed in PBS for 30 min. Sections were then incubated in permeabilization solution (0.1% Triton X-100 and 0.1% sodium citrate in PBS) for 2 minutes on ice. 50 μL of TUNEL reaction mixture were then added to samples and incubated at 37° C. for 1 hr. After washing in PBS (2 times, 5 min), nuclei were counterstained with NucBlue (Thermo Fisher Scientific) for 15 min at room temperature. Following final washes (PBS, three times for 5 min), glass coverslips were added to the sections using Fluoroshield. A TCS SP5 AOBS laser scanning confocal microscope (Leica Microsystems) was used for imaging the stained microtissue sections.

mRNA isolation, cDNA synthesis, and Real Time-Quantitative Polymerase Chain Reaction (RT-qPCR) analysis. For RT-qPCR analysis, mRNA was isolated using the E.N.Z.A. Total RNA Isolation kit I (Omega Bio-Tek, Norcross, GA, USA) according to the manufacturer's instructions. Briefly, hPSC cardiac organoids were lysed using TRK Lysis buffer supplemented with 1:1000 dilution of β-mercaptoethanol (Sigma-Aldrich). Solutions of lysed organoids were homogenized using the Omega Homogenizer spin columns. mRNA was then bound to HiBind mRNA binding columns and rinsed once with Wash Buffer I and twice with Wash Buffer II. mRNA was eluted in RNAse-, DNAse-Free DI water (Thermo Fisher Scientific) and measured using a NanoDrop One Microvolume UV-Vis Spectrophotometer (Thermo Fisher Scientific). For cDNA synthesis, the iScript cDNA synthesis kit (Bio-Rad, Hercules, CA, USA) was used according to the manufacturer's instructions using an Axygen MacGENE II Thermocycler (Thermo Fisher Scientific). RT-qPCR was performed using the Life Technologies Taqman probe system. 25 ng of cDNA templates were mixed with 2X Applied Biosystems Universal PCR Mastermix (Thermo Fisher Scientific) and Taqman primers and probes (Thermo Fisher Scientific) according to the manufacturer's instructions and ran on CFX96 Real Time PCR detection machines (Bio-Rad). mRNA expression per gene was expressed as 2^{-ddCt}. First, data was normalized as the difference in cycle threshold (Ct) of the gene of interest to the housekeeping gene, GAPDH, (ΔCt) and then normalized per experimental group by taking the difference in ΔCt of the sample of interest to the negative control (AACt) and reported as the mRNA relative expression (2^{-ΔΔCt).}

Single-cell RNA sequencing. Day 5 3- and 4-cell cardiac organoids were dissociated via treatment with 1X TrypLE (Thermo Fisher Scientific) for 40 minutes at 37° C., 5% CO₂, pipetting the cell solution every 5 minutes. We prepared libraries according to the Chromium NextGEM Single Cell 3′ Reagent Kit (10X Genomics, Pleasanton, CA, USA). Libraries were sent to Azenta Life Sciences for sequencing on the Illumina NovaSeq600 platform and were run through the 10X Genomics CellRanger pipeline. Further analyses within Python (v.3.12.7) using the Scanpy package (v1.10.2). AnnData objects for each library were merged, then integrated using the Harmony package (v1.2.1). Annotation was conducted using canonical markers for cardiomyocytes, fibroblasts, endothelial cells, and pericytes.

Rat model of ischemia-reperfusion injury. Procedures for our rat model of ischemia-reperfusion injury were similar to those previously described in¹. However, in this study, we used ketamine and xylazine for anesthesia instead of isoflurane to reduce post-operation mortality. Briefly, male athymic rats (8 to 10 weeks old, RNU Nude rats, Charles River) were anesthetized with ketamine and xylazine, intubated, and mechanically ventilated (SomnoSuite Low-Flow Anesthesia System, Kent Scientific). Rodent Surgical Monitor (INDUS instruments) was used to monitor the animal's condition throughout the surgery, including heart rate, body temperature, and heart function through ECG. A left thoracotomy was performed to expose the heart. The pericardium was opened, and the left anterior descending coronary artery was ligated below the left atrial appendage level using a 7-0 Prolene suture (Ethicon). Occlusion was confirmed by LV blanching and ST elevation on ECG after ligation. After 1 h, the ligation was released, the chest cavity was aseptically closed, and the rat was allowed to recover for 24 h for echocardiographic measurement. For Day 28 studies, the rats that met the echocardiographic inclusion criterion (FS <30%) were selected for experiments and randomly assigned into experimental groups.

Injection of 4-cell hPSC organoids into injured rat hearts. Procedures for hPSC organoid injection were based on those described in¹. In brief, we injected organoids 4 days after I/R induction to allow angiogenesis-associated wound healing processes to reach their peak⁶. The rats that met our inclusion criterion were anesthetized with ketamine and xylazine, intubated, and mechanically ventilated. A second thoracotomy was performed to expose the heart. In preparation for injection, one-third of the total number of cardiac organoids was manually loaded into each of the three syringes with a Geltrex (Thermo Fisher Scientific) plug, which was used to occupy the dead space in the syringe for organoid injection and plug the injection path to prevent washout of organoids. A suspension of 4-cell hPSC cardiac organoids containing a total of 1.4×10⁶ total cells were injected with a modified 29-gauge needle into the region surrounding the injured area at three sites (50 μL per site). I/R-injured rats without the cell injection were used as controls and did not receive a second surgery. Once the injections were completed, the chest was closed in three layers, and the animal was recovered with buprenorphine administered intraperitoneally to provide pain relief.

Echocardiography. Echocardiography was performed as described in¹. Briefly, transthoracic echocardiography was performed using Vevo2100 ultrasound imaging system (VisualSonics, Toronto, Canada) with a 13 to 24 MHz linear array transducer (MS250). Rats were placed in supine position on a warming platform at 37° C. under light (1 to 2%) isoflurane anesthesia to maintain the heart rate between 350 and 450 beats/min during imaging procedure. Cardiac function was examined using parasternal long-axis and short-axis 2D B-mode images and M-mode images at parasternal short-axis mid-papillary level. Offline image analyses were performed by an experienced investigator blind to the experimental groups using the dedicated Vevo LAB software. FS was calculated by the equation: FS=(LV end-diastolic dimension−LV end-systolic dimension)/LV end-diastolic dimension.

Histological evaluation of harvested rat hearts. The following procedures were performed as described in 1. In brief, excised rat hearts were fixed overnight in 4% paraformaldehyde solution (Sigma Aldrich) and then to 70% ethanol before paraffin embedding. Using an automated microtome (LeicaRM2255, Leica, Exton, PA), paraffin-embedded hearts were sectioned at 8 μm in thickness. Slides were deparaffinized in xylene for 5 min and rehydrated using an ethanol gradient. Slides were then stained with hematoxylin and eosin (H&E) and picrosirius red stain kit (Abcam) or processed with heat-activated antigen retrieval solution (Vector Laboratories, Burlingame, CA) for 5 minutes for immunostaining as detailed below. H&E staining was performed every 100 μm to identify organoid grafts. Sections for immunostaining (described below) were selected as adjacent slides to H&E-identified organoid grafts. For percent fibrosis and wall thickness calculations, three regions from base to apex in each heart were used to quantify averages.

Immunostaining of rat hearts. Immunostaining was performed as described previously¹. Briefly, following heat-activated antigen retrieval, fixed rat heart sections were washed with PBS, permeabilized with 0.1% Triton X-100 (Sigma-Aldrich) in PBS (PBST-0.1) and blocked with 10% Goat serum (Sigma-Aldrich) for 2 h at room temperature. Primary antibodies were applied overnight, and secondary antibodies were applied for 1 h at room temperature, with 3, 10-min PBST-0.1 washes between primary and secondary stains. Primary antibodies used: rabbit anti-TnI (Invitrogen) and mouse anti-HNA (Abcam). Secondary antibodies used: goat anti-rabbit Alexa Fluor 647 (Invitrogen) and goat anti-mouse Alexa Fluor 488 (Invitrogen). Additionally, isolectin Alexa Fluor 488 conjugate (IB4) (Vector Laboratories) was used for staining rat vasculature. Nuclei were counterstained with DAPI (Molecular Probes/Invitrogen, Eugene, OR) diluted in PBST for 30 min at room temperature. Following the final wash procedure (PBST-0.1, three times for 5 min), glass coverslips were added to the slides using Fluoroshield. Lastly, a TCS SP5 AOBS laser scanning confocal microscope (Leica Microsystems Inc., Exton, PA) was used to acquire fluorescent images.

Evaluation of cardiac hypertrophy. Cardiac hypertrophy measurements were performed as described previously¹. In brief, cardiomyocyte cross-sectional area was determined via staining of cell boundaries with Alexa Fluor 488-conjugated WGA (Invitrogen). Slides were incubated with WGA (10 μg/ml) for 20 min at room temperature and washed with PBS three times before mounting coverslips. Three regions from base to apex in each heart were used to quantify average cross-sectional area per heart. To quantify the cell size, three independent samples per group with three different regions of interest from the border and remote zones in the left ventricle were captured at 40× magnification with a TCS SP5 AOBS laser scanning confocal microscope (Leica Microsystems). Images were binarized in ImageJ and the Analyze Particles function was used to quantify individual cardiomyocyte area.

Statistical analysis. Differences between experimental groups were analyzed using Microsoft Excel (v. 16) and GraphPad Prism (v. 10) using two-tailed Student's t-test or one-way ANOVA with Tukey's multiple comparisons. Statistical difference was considered with p-value<0.05.

### Results and Discussion

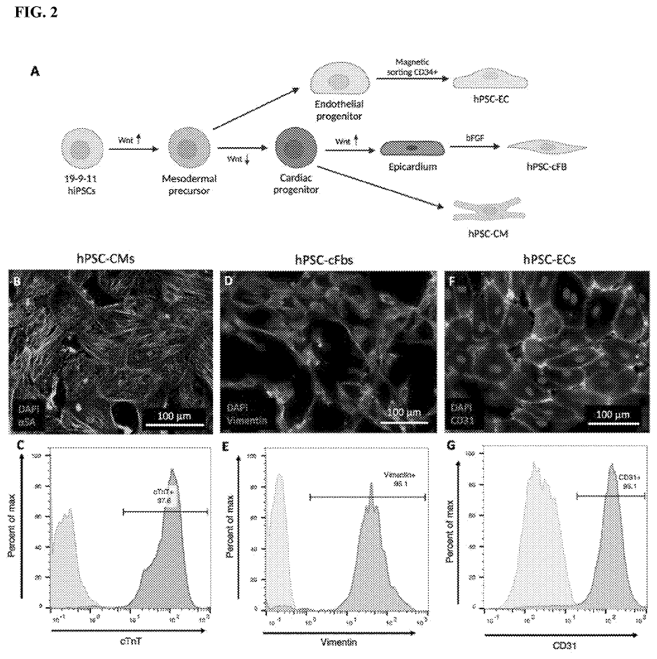

Differentiation of hPSC-derived cardiomyocytes, fibroblasts, and endothelial cells from 19-9-11 hiPSCs. Our first step in developing isogenic hPSC-derived cardiac organoids was differentiating hPSC-CMs, hPSC-cFbs, and hPSC-ECs from one hPSC line (19-9-11 hiPSCs) (FIG. 2, Panel A). hPSC-CM differentiation followed the GiWi protocol, which includes activation of the Wnt pathway via GSK inhibition (CHIR99021 treatment) followed by inhibition of the Wnt pathway (IWP2 treatment) to produce cardiac progenitor cells on Day 6 of the protocol^{33,46}. Correct conditions for stem cell differentiation can vary depending on cell line and small molecule lot³³. Therefore, the differentiation of hPSC-CMs was accompanied by optimization of initial seeding density and concentration of CHIR99021 for mesodermal induction. On Day 10, we observed spontaneous contraction in our wells of optimally differentiated hPSC-CMs, although at this stage, cultures consisted of both cardiomyocytes and nonmyocyte cells. Treatment of impure hPSC-CM cultures with lactate purification media allowed elimination of non-cardiomyocyte cells due to their inability to metabolize lactate⁴⁷. After establishing optimal differentiation protocols to produce pure cultures of hPSC-CMs, we successfully and robustly observed hPSC-CMs of expected phenotype, confirmed by expression of a-sarcomeric actinin via 2D immunofluorescent staining (FIG. 2, Panel B) and >95% expression of cardiac troponin T (cTnT) via flow cytometry (FIG. 2, Panel C).

For the differentiation of hPSC-cFbs we followed protocols established for epicardial-derived cardiac fibroblasts^{37,48,49}. For this differentiation, hPSCs are differentiated into hPSC-derived epicardial cells before differentiation into hPSC-cFbs. We chose to perform hPSC-cFb differentiation and culture on decellularized heart extracellular matrix (HEM) due to its capacity to alleviate activation of hPSC-cFbs³². Characterization of hPSC-cFbs revealed high expression of vimentin-positive cells, as shown by 2D immunofluorescent staining (FIG. 2, Panel D) and flow cytometry (FIG. 2, Panel E).

hPSC-ECs were differentiated in following a combination of protocols^{42, 43, 50, 41} in combination. For endothelial cells derived from the 19-9-11 hiPSC line, we found that magnetic-activated cell sorting (MACS) of CD34+ cells on Day 5 followed by culture in endothelial growth media-2 (EGM 2) supplemented with TGF inhibitor (0.5 μM A83-01) for 5 additional days most efficiently yielded high purity and highly proliferative cells. Inhibiting TGFβ has been shown to induce proliferation in hPSC-ECs51 and proved to be vital in driving prolonged culture of our cells. At Day 10 of the protocol, we observed highly pure populations of CD31+hPSC-ECs, validated by 2D cell staining (FIG. 2, Panel F) and flow cytometry (FIG. 2, Panel G). Taken together, the data illustrates successful differentiation of hPSC-CMs, hPSC-cFbs, and hPSC-ECs from the 19-9-11 hiPSC line.

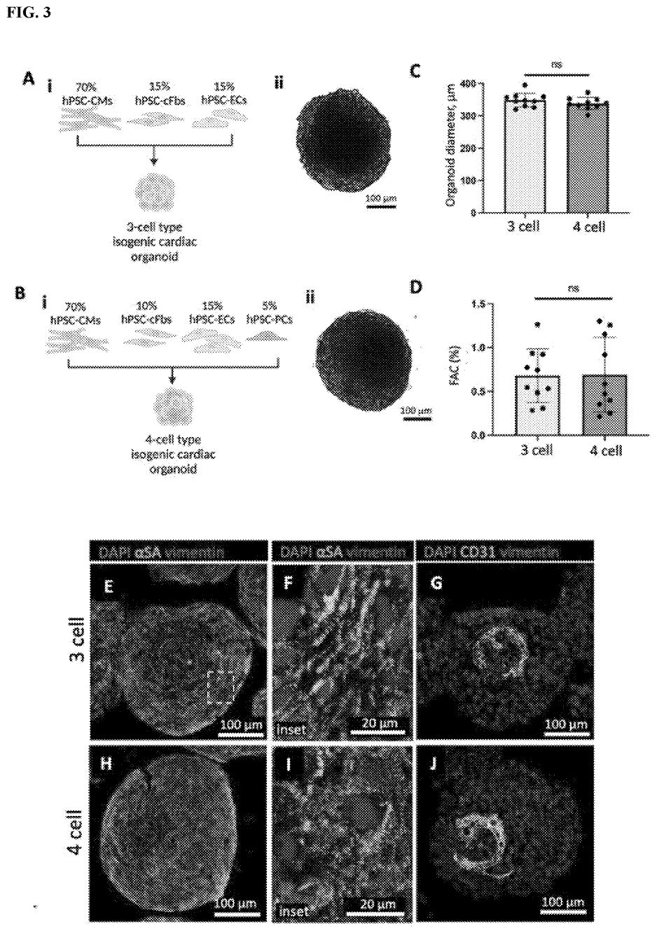

Fabrication of 3- and 4-cell type isogenic hPSC-derived cardiac organoids. Upon differentiation of hPSC-CMs, hPSC-cFbs, and hPSC-ECs from a common hiPSC line, we began developing isogenic cardiac organoids. We initially explored 3-cell type cardiac organoids composed of cells at a ratio of 70% hPSC-CMs: 15% hPSC-cFbs: 15% hPSC-ECs (“3-cell type cardiac organoids”, FIG. 3, Panel A (i)). This ratio mimics that of cells in the developing heart, which contains 75-80% cardiomyocytes and 20-25% nonmyocyte cells⁵². We found that cardiac organoids of this composition robustly self-assembled into spontaneously contracting spherical aggregates by Day 5 (FIG. 3, Panel A (ii)). We desired to investigate the incorporation of hPSC-PCs in our isogenic cardiac organoids to induce optimal hPSC-ECs expression and organization and enhance organoid prevascularization. Therefore, we first needed to confirm the capacity to differentiate hPSC-PCs from the 19-9-11 hiPSC line. Based on the protocol established by Faal et al. to produce mesodermal-derived pericytes⁴⁴, we successfully differentiated hPSC-PCs of the expected, marked by positive expression of NG2 and CD13 and negative expression of CD34. We next modified the composition of cells in our 3-cell type cardiac organoids to introduce hPSC-PCs by allocating 5% of the total cell composition to hPSC-PCs and decreasing the ratio of hPSC-cFbs to 10%, as the ratio of PCs to ECs in the coronary vasculature is between 1:2 and 1:353(“4 -cell type cardiac organoids”, FIG. 3, Panel B (i)). We found that 4-cell type organoids containing hPSC-PCs also formed spontaneously contracting spherical microtissues at Day 5, similar to 3-cell type organoids (FIG. 3, Panel B (ii)).

Furthermore, we saw that the incorporation of hPSC-PCs did not affect the organoid diameter (FIG. 3, Panel C) nor the contractile ability of the organoids, measured by fractional area change (FAC) (FIG. 3, Panel D). Additionally, we noted that the inclusion of hPSC-cFbs was vital for hPSC-EC organization in both 3- and 4-cell type organoids. Without hPSC-cFbs in 3-cell type organoids (i.e., 70% hPSC-CMs and 30% hPSC-ECs), hPSC-ECs are unable to integrate with hPSC-CMs. The lack of hPSC-cFbs in 4-cell type organoids (i.e., 70% hPSC-CMs, 15% hPSC-ECs, 15% hPSC-PCs) yields organoids with minimal expression of CD31+ positive cells. Both 3- and 4-cell type cardiac organoids exhibited similar overall composition, with the presence of hPSC-CMs along the peripheral region of the organoids, marked by αSA expression (FIG. 3, Panels E-F, H-I), and organized networks of hPSC-ECs toward the center of the organoid, designated by CD31 expression (FIG. 3, Panels G, J). The organization of hPSC-CMs and hPSC-ECs followed expected organization observed in our primary cell organoids in which hPSC-CMs organize at the periphery and hPSC-ECs migrate toward the center. We hypothesize that this is due to the oxygen gradient present in microtissues of ~200 μm in diameter^{24,45}. As a whole, our data demonstrates that hPSC-derived cells from a common hiPSC line can self-assessable into spontaneously contracting microtissues.

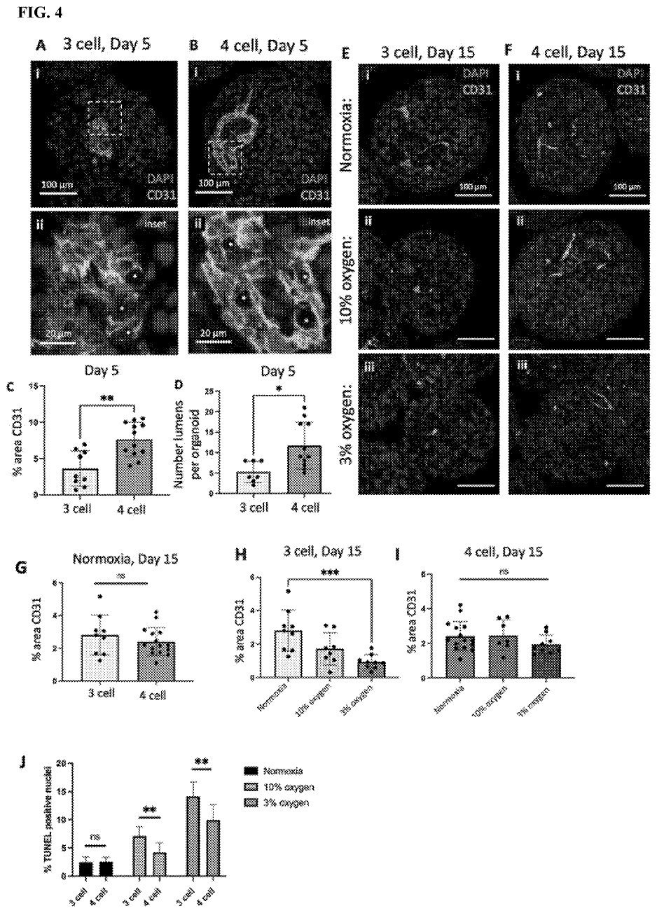

Support of vascular development and survival in normoxia and hypoxia with the inclusion of hPSC-PCs. Due to the role pericytes play in endothelial cell stability and organization, we explored the impacts of incorporating hPSC-PCs in 4-cell type cardiac organoids on hPSC-ECs. We observed that on Day 5, hPSC-ECs in both 3- and 4-cell type cardiac organoids created CD31+ networks of cells with lumenized structures throughout (FIG. 4, Panels A-B). However, we observed significantly increased area of CD31+ cells in 4-cell type cardiac organoids (FIG. 4, Panel C) as well as increased number of lumens per organoid (FIG. 4, Panel D) on Day 5.

We next wanted to investigate the effects of hypoxia on organoid structure and cell survival as the ultimate goal of this study was to test the therapeutic efficacy of cardiac organoids in infarcted rat hearts. Following 10 days of culture in hypoxic conditions, we saw that the expression of CD31+ stayed constant between 3- and 4-cell type organoids in normoxia; however, the CD31+ expression significantly decreased in 3% oxygen conditions in 3-cell type organoids (FIG. 4, Panels E-I). This suggests that the inclusion of hPSC-PCs protects hPSC-ECs in hypoxic conditions. We next sought to investigate the impacts of incorporating hPSC-PCs on overall cell survival in hypoxic conditions. We saw that the inclusion of hPSC-PCs (i.e., 4-cell type organoids) yields organoids with overall decreased apoptosis, marked by significantly less TUNEL positive nuclei in 4-cell type cardiac organoids after culture in both 10% and 3% oxygen (FIG. 4, Panel J). Taken together, our data suggests that including hPSC-PCs in cardiac organoids both drives hPSC-EC expression in normoxia and hypoxia and contributes to promoting cell survival following hypoxic culture.

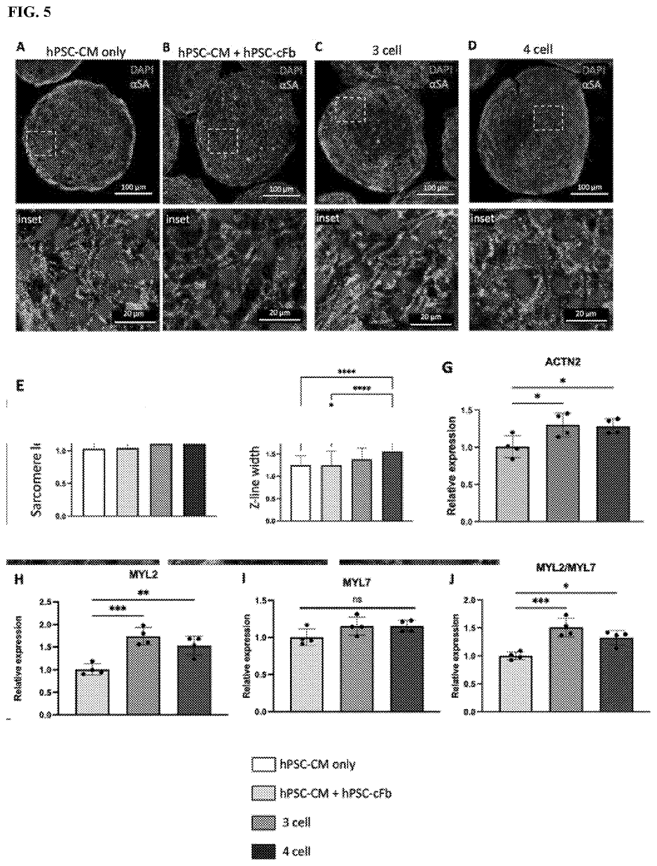

Enhancement of sarcomere development and expression in normoxia and hypoxia. Endothelial cells throughout the coronary vasculature are in close contact with all cells in the heart, specifically cardiomyocytes, and provide multiple signals to regulate function⁵⁴. As we have demonstrated that the incorporation of hPSC-PCs enhances hPSC-ECs expression, we investigated the impacts of improved vascular network formation on other cell types in the isogenic cardiac organoids, specifically hPSC-CMs. We compared sarcomere expression and development in microtissues of different supporting cell-type compositions: hPSC-CM only spheroids (FIG. 5, Panel A), 70% hPSC-CM: 30% hPSC-cFbs (FIG. 5, Panel B), 3-cell type organoids (FIG. 5, Panel C), and 4-cell type organoids (FIG. 5, Panel D). While there was no impact in including different supporting cells on sarcomere length (FIG. 5, Panel E), we observed significant enhancement in z-line width with both the inclusion of hPSC-ECs (i.e., 3-cell type organoids) and most significantly with hPSC-PCs in addition to hPSC-ECs (i.e., 4-cell type organoids) (FIG. 5, Panel F). We next investigated transcriptomic differences in organoids that could contribute sarcomere expression. We observed increased expression of ACTN2 and MYL2 with the inclusion of hPSC-ECs (FIG. 5, Panels G-H). We did not see enhanced expression in MYL7 (FIG. 5, Panel I) across organoids but did observe significant increase in the ratio of MYL2/MYL7 (FIG. 5, Panel J), suggesting a shift of hPSC-CMs in 3- and 4-cell type cardiac organoids toward a ventricular phenotype. Taken together, this data suggests that the incorporation of hPSC-ECs as a whole and the increased expression of hPSC-ECs in 4-cell type cardiac organoids correlates with increased hPSC-CM development.

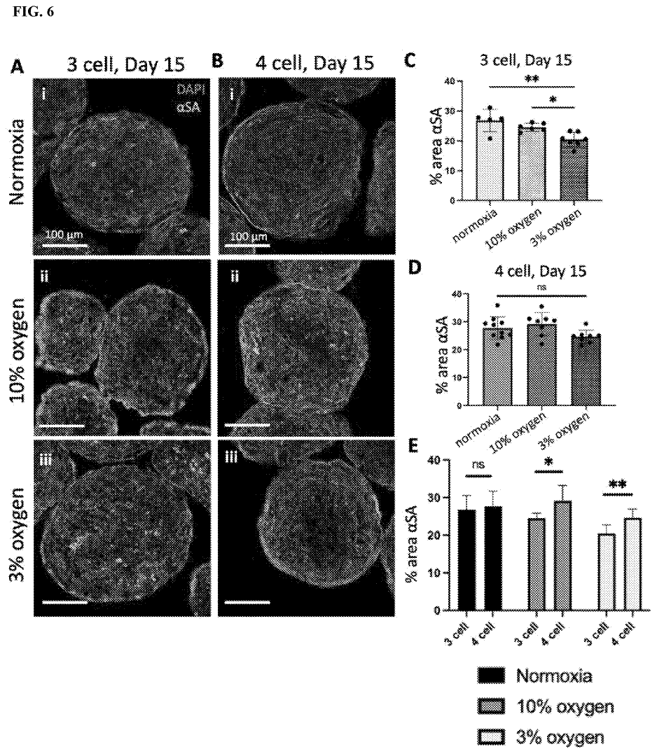

We next sought to examine the effects of hPSC-PC incorporation on the presence of hPSC-CMs after 10 days of hypoxic culture. We cultured 3-cell (Panel. 6, Panel A) and 4-cell (FIG. 6, Panel B) cardiac organoids in conditions of normal oxygen, 10% oxygen, and 3% oxygen. We found that the presence of hPSC-CMs, marked by a-SA expression, in 3-cell type organoids significantly diminished in 3-cell type organoids in hypoxic conditions (FIG. 6, Panel C); whereas, in 4-cell type organoids, the presence of hPSC-CMs stayed constant (FIG. 6, Panel D). Furthermore, the percent expression of α-SA was significantly higher in 4-cell type organoids compared to 3-cell type organoids in both 10% and 3% oxygen conditions. This data suggests that the incorporation of hPSC-PCs in 4-cell type organoids correlates with enhanced hPSC-CM presence in hypoxic conditions.

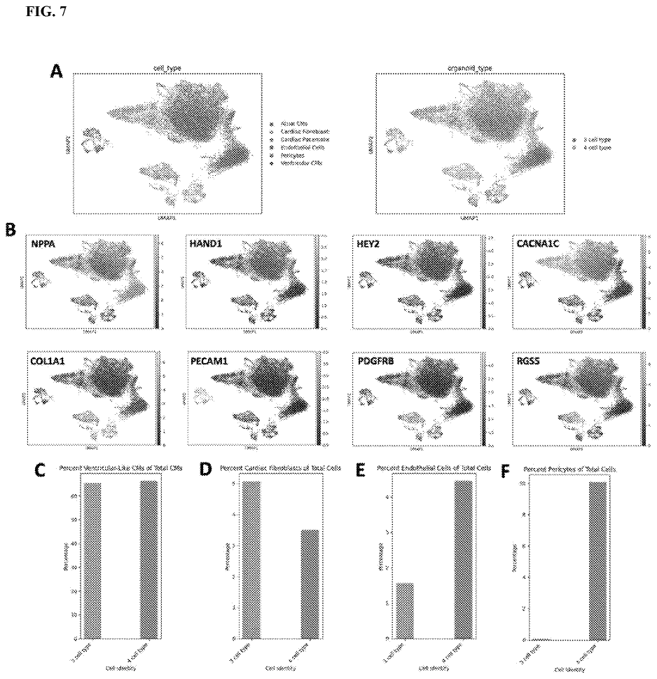

Comparison of 3-cell type and 4-cell type isogenic cardiac organoids via single-cell RNA sequencing. To obtain a more thorough understanding of specific cell types present in hPSC cardiac organoids and the effects of incorporating hPSC-PCs on each cell type in the isogenic cardiac organoids, we performed single-cell RNA sequencing (scRNA-seq). Following dissociation of 3- and 4-cell type organoids to single cells, we prepared libraries for sequencing. An exploration of gene expression in each cluster allowed identification of each cell type included in our cardiac organoids, including cardiomyocyte subtypes, endothelial cells, fibroblasts, and pericytes (FIG. 7, Panels A-B). Interestingly, we observed a population of cells unique to 4-cell type organoids with increased expression of PDGFRB and RGS5, which are both characteristic of pericyte transcriptome. Furthermore, we investigated the percent of total cell composition by each cell type in 3- and 4-cell type cardiac organoids. The percent of ventricular-like hPSC-CMs was similar in 3- and 4-cell types (FIG. 7, Panel C). This was expected due to the fact that the percent of hPSC-CMs in 3- and 4-cell type cardiac organoids was the same upon fabrication (70% hPSC-CMs). The percent of hPSC-cFbs was lower in 4-cell type cardiac organoids (FIG. 7, Panel D) which was expected due to the fact that the original composition of 3-cell type cardiac organoids contained 15% hPSC-cFbs while the 4-cell type cardiac organoids contained 10% hPSC-cFbs. Interestingly, although we fabricated both 3- and 4-cell type cardiac organoids with 15% hPSC-ECs, we observed that the percent of hPSC-ECs as determined by scRNA-seq was higher in 4-cell type cardiac organoids compared to that of 3-cell type cardiac organoids at Day 5 (FIG. 7, Panel E). This validated our earlier findings of increased percent expression of CD31+ cells in 4-cell type cardiac organoids via immunofluorescent staining (FIG. 4, Panels A-C). In terms of percent of hPSC-PCs in 3-cell type cardiac organoids, we saw essentially no presence of hPSC-PCs in 3-cell type cardiac organoids, as expected (FIG. 7, Panel F).

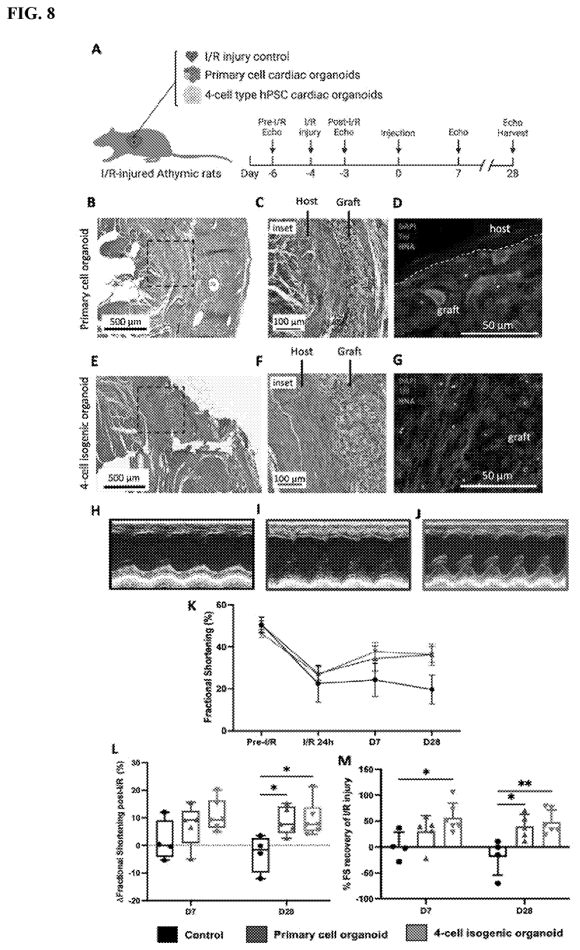

Functional engraftment of 4-cell type isogenic organoids in infarcted myocardium. After establishing that 4-cell type cardiac organoids have enhanced hPSC-EC and hPSC-CM development, specifically in hypoxic conditions, we moved forward with injecting 4-cell type organoids into a rat model of I/R injury to test their therapeutic efficacy. To ensure that our transition from primary cell cardiac organoids to isogenic cardiac organoids yielded similar amounts of functional improvement of injure rat hearts, we compared 4-cell cardiac organoid injection to previously published data of primary cell cardiac organoid injection and I/R injury control rats¹. We injected 4-cell cardiac organoids 4 days after I/R injury in athymic rats, performing echocardiography before I/R injury (pre-I/R), 24 h after injury (post-I/R), 7 days after injection, and 28 days post-injection (FIG. 8, Panel A). We established an inclusion criteria of <30% fractional shortening (FS) post-I/R. We injected approximately 500 organoids per rat for a total number of ~1×10⁶ total hPSC-CMs and ~1.5×10⁶ total cells per rat. At Day 28, we observed engraftment of 4-cell cardiac organoids, similar in size to that of primary cell organoids (FIG. 8, Panels B-C, E-F). We confirmed graft presence by immunofluorescent staining of slow-skeletal troponin I (TnI), a protein found primarily in immature cardiomyocytes (i.e., hPSC-CMs) and not in adult cardiomyocytes⁵⁵, and human nucleolar antigen (HNA) (FIG. 8, Panels D, G). Furthermore, we saw development of sarcomeric structures via TnI staining in both primary cell and 4-cell type organoid engraftments (FIG. 8, Panels D, G). Upon echocardiographic measurements, we observed reduced left ventricular (LV) wall motion in control groups (FIG. 8, Panel H) compared to that of primary cell and 4-cell organoid-treated groups (FIG. 8, Panels I-J). All groups exhibited a severe reduction in LV function post-I/R and had similar post-I/R FS measurements. However, upon treatment following I/R injury, FS increased in groups treated with both primary cell organoids and 4-cell organoids (FIG. 8, Panel K). By Day 28, rats treated with both primary cell and 4-cell cardiac organoids demonstrated significantly higher FS increase over the post-I/R value (FIG. 8, Panel L) as well as significantly increased percent recovery of FS from I/R injury (FIG. 8, Panel M). Of note, 4-cell cardiac organoids promoted significantly increased percent recovery of FS at both Day 7 and Day 28 compared to control rats (FIG. 8, Panel M). As a whole, the data suggests that 4-cell cardiac organoids are similarly efficacious in promoting functional recovery of I/R injured rat hearts as compared to primary cell organoids.

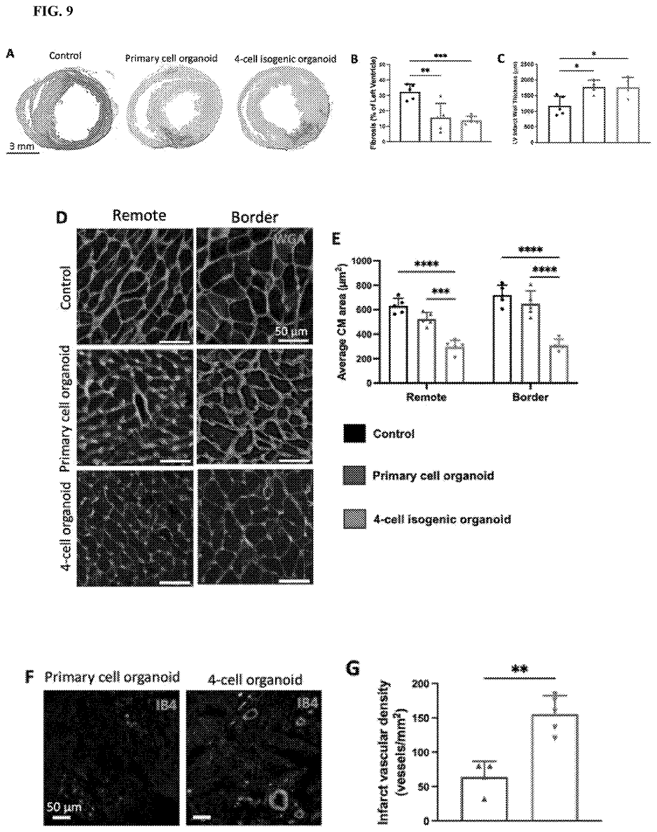

4-cell type isogenic organoids mitigate adverse left ventricular remodeling after I/R injury. Hallmarks of pathological ventricular remodeling after myocardial infarction include cardiac fibrosis, LV wall thinning, and cardiomyocyte hypertrophy³⁶. Therefore, we examined the effects of injecting 4-cell cardiac organoids on LV remodeling via measurement of cardiac fibrosis and LV infarct wall thickness at Day 28. We chose Day 28 for assessment of pathological ventricular remodeling due to the fact that this timepoint would allow evaluation of both the acute and chronic inflammatory response to injury. Using Picrosirius Red staining, we identified collagenous scar tissue in all groups in the LV due to I/R injury (FIG. 9, Panel A). However, we found that both primary cell and 4-cell cardiac organoids had similar significantly decreased levels of fibrotic tissue occupying the LV compared to control rat hearts at similar levels (FIG. 9, Panel B). Additionally, we found that both primary cell and 4-cell isogenic cardiac organoids had significantly less LV wall thinning at the infarct site compared to controls (FIG. 9, Panel C).

We also investigated the extent of cardiac hypertrophy in the remote and border regions of control and organoid-treated hearts via wheat germ agglutinin (WGA) staining of host cardiomyocyte membranes (FIG. 9, Panel D). Compared to control, the area of host cardiomyocytes in the remote zone of 4-cell cardiac organoids were significantly lower than those of both control and primary cell-treated rats (FIG. 9, Panel E). In the border region, the cross-sectional area of host cardiomyocytes was significantly lower in 4-cell organoid-treated rats than both control and primary-cell treated rat hearts, suggesting increased attenuation of cardiac hypertrophy (FIG. 9, Panel E). Upon measurement of arteriole density at the infarct site (FIG. 9, Panel F), we observed that injection of 4-cell cardiac organoids promotes increased infiltration of host vasculature to the infarct region (FIG. 9, Panel G).

Taken together, this data suggests that our 4-cell type cardiac organoid is as effective in attenuating adverse left ventricular remodeling as primary cell cardiac organoids. In terms of cardiac hypertrophy and promoting therapeutic angiogenesis post-I/R, 4-cell cardiac organoids elicit increased therapeutic effects compared to control and primary-cell treated rat hearts (FIG. 9, Panels E, G). This data supports our findings of improved heart function in organoid-treated rat hearts compared to control (FIG. 8).

### Conclusions

In this study, we successfully and reproducibly differentiated hPSC-CMs, hPSC-cFbs, and hPSC-ECs from the 19-9-11 hiPSC line, as demonstrated by their respective expression of a-sarcomeric actinin (αSA) and cardiac troponin T (cTnT), vimentin, and CD31. We demonstrated their capacity to self-assemble to form spontaneously contracting microtissues and further improved their structure with the inclusion of hPSC-PCs. We observed that hPSC-derived organoids with hPSC-PCs (4-cell type cardiac organoids) had enhanced vascular network development as well as cell survival and sarcomere structure in normoxia and hypoxia. We therefore progressed with 4-cell type cardiac organoids for transplantation in our rat model of I/R injury. We found that 4-cell type cardiac organoids were as effective as our previous primary cell cardiac organoid model in promoting functional recovery in injured rat hearts. Furthermore, we observed that 4-cell type organoids were capable of mitigating adverse LV remodeling following ischemic injury. Taken together, we demonstrated an effective transition from primary cell cardiac organoids to isogenic, hPSC-derived cardiac organoids as cardiac regenerative therapies.

In future studies, we would like to investigate the direct and indirect mechanisms through which hPSC-PCs contribute to the enhancements in cardiac organoids. We believe that targeting N-cadherin and Notch signaling would be instrumental in studying the direct effect effects of hPSC-PCs in organoid enhancements due to their roles in the interactions between pericytes and endothelial cells^{57,58}. Disruption in the presence of N-cadherin leads to vessel abnormalities and suggests a critical role of N-cadherin in cell-cell adhesion between endothelial and mural cells^{59,60}. One possible manner to investigate the roles of N-cadherin and Notch signaling in the interactions between hPSC-PCs and hPSC-ECs in our cardiac organoids would be the development of Notch and N-cadherin knockdowns via transfection of hPSC-PCs with antisense oligonucleotide Gapmers. We also believe targeting the PI3k-Akt pathway in cardiac organoids could give useful insights into the observed enhancement of cell survival in hypoxia. PI3k activation has been shown to be associated with improved cell survival⁶¹, and PI3k inhibitors, such as LY294002, have been used to demonstrate that cardioprotective effects are mediated via the PI3k-AKT pathway⁶². Furthermore, our scRNA-seq libraries would be of great benefit in studying the direct and indirect roles of hPSC-PCs. Our libraries allow us to compare distinct differences between cells in 3- and 4-cell type organoids. We will be able to designate clear enhancements in each cell population in comparison to its 3-cell type counterpart. Additionally, a useful tool for analyzing our sequencing data will be CellChat, which allows inference regarding intercellular communication pathways from scRNA-seq data⁶³. CellChat has been implemented in exploring cell-cell interactions in human heart tissue⁶⁴ as well as identifying interactions between hPSC-CM and hPSC-EC in 3D cardiac microtissues⁶⁵.

We also believe it would be of interest to investigate the effects of the stress conditions of the ischemic rat heart on cardiac organoids via in vitro testing. In previous publications, we have mimicked the ischemic environment by culturing cardiac organoids in 1% oxygen with 10X diluted maintenance media¹. Furthermore, we believe our in vivo studies could be further improved with investigation of the innate immune response to cardiac organoid injections, including macrophage infiltration and polarization in engrafted rat hearts.

### REFERENCES

1 Tan, Y., Coyle, R. C., Barrs, R. W., Silver, S. E., Li, M., Richards, D. J., Lin, Y., Jiang, Y., Wang, H., Menick, D. R., Deleon-Pennell, K., Tian, B. & Mei, Y. Nanowired human cardiac organoid transplantation enables highly efficient and effective recovery of infarcted hearts. Sci Adv. 2023;9(31): eadf2898. Epub 2023 Aug. 4. doi: 10.1126/sciadv.adf 2898. PubMed PMID: 37540743; PubMed Central PMCID: PMCPMC10403216.

2 Garbern, J. C., Escalante, G. O. & Lee, R. T. Pluripotent stem cell-derived cardiomyocytes for treatment of cardiomyopathie damage: Current concepts and future directions. Trends Cardiovasc Med. 2021;31(2):85-90. Epub 2020 Jan. 28. doi: 10.1016/j.tcm.2020.01.002. PubMed PMID: 31983535.

3 Funakoshi, S., Miki, K., Takaki, T., Okubo, C., Hatani, T., Chonabayashi, K., Nishikawa, M., Takei, I., Oishi, A., Narita, M., Hoshijima, M., Kimura, T., Yamanaka, S. & Yoshida, Y. Enhanced engraftment, proliferation, and therapeutic potential in heart using optimized human iPSC-derived cardiomyocytes. Scientific reports. 2016;6:19111. Epub 2016 Jan. 9. doi: 10.1038/srep19111. PubMed PMID: 26743035; PubMed Central PMCID: PMC4705488.

4 Caspi, O., Huber, I., Kehat, I., Habib, M., Arbel, G., Gepstein, A., Yankelson, L., Aronson, D., Beyar, R. & Gepstein, L. Transplantation of human embryonic stem cell-derived cardiomyocytes improves myocardial performance in infarcted rat hearts. Journal of the American College of Cardiology. 2007;50(19): 1884-1893. Epub 2007 Nov. 6. doi: 10.1016/j.jacc.2007.07.054. PubMed PMID: 17980256.

5 Guan, X., Xu, W., Zhang, H., Wang, Q., Yu, J., Zhang, R., Chen, Y., Xia, Y., Wang, J. & Wang, D. Transplantation of human induced pluripotent stem cell-derived cardiomyocytes improves myocardial function and reverses ventricular remodeling in infarcted rat hearts. Stem Cell Res Ther. 2020;11(1):73. Epub 2020 Feb. 23. doi: 10.1186/s13287-020-01602-0. PubMed PMID: 32085809; PubMed Central PMCID: PMCPMC7033912.

6 Laflamme, M. A., Chen, K. Y., Naumova, A. V., Muskheli, V., Fugate, J. A., Dupras, S. K., Reinecke, H., Xu, C., Hassanipour, M., Police, S., O'Sullivan, C., Collins, L., Chen, Y., Minami, E., Gill, E. A., Ueno, S., Yuan, C., Gold, J. & Murry, C. E. Cardiomyocytes derived from human embryonic stem cells in pro-survival factors enhance function of infarcted rat hearts. Nature biotechnology. 2007;25(9):1015-1024. Epub 2007 Aug. 28. doi: 10.1038/nbt1327. PubMed PMID: 17721512.

7 Shiba, Y., Fernandes, S., Zhu, W. Z., Filice, D., Muskheli, V., Kim, J., Palpant, N. J., Gantz, J., Moyes, K. W., Reinecke, H., Van Biber, B., Dardas, T., Mignone, J. L., Izawa, A., Hanna, R., Viswanathan, M., Gold, J. D., Kotlikoff, M. I., Sarvazyan, N., Kay, M. W., Murry, C. E. & Laflamme, M. A. Human ES-cell-derived cardiomyocytes electrically couple and suppress arrhythmias in injured hearts. Nature. 2012;489(7415):322-325. Epub 2012 Aug. 7. doi: 10.1038/nature11317. PubMed PMID: 22864415; PubMed Central PMCID: PMC 3443324.

8 Kawamura, M., Miyagawa, S., Miki, K., Saito, A., Fukushima, S., Higuchi, T., Kawamura, T., Kuratani, T., Daimon, T., Shimizu, T., Okano, T. & Sawa, Y. Feasibility, safety, and therapeutic efficacy of human induced pluripotent stem cell-derived cardiomyocyte sheets in a porcine ischemic cardiomyopathy model. Circulation. 2012; 126(11 Suppl 1):S29-37. doi: 10.1161/CIRCULATIONAHA.111.084343. PubMed PMID: 22965990.

9 Ishida, M., Miyagawa, S., Saito, A., Fukushima, S., Harada, A., Ito, E., Ohashi, F., Watabe, T., Hatazawa, J., Matsuura, K. & Sawa, Y. Transplantation of Human-induced Pluripotent Stem Cell-derived Cardiomyocytes Is Superior to Somatic Stem Cell Therapy for Restoring Cardiac Function and Oxygen Consumption in a Porcine Model of Myocardial Infarction. Transplantation. 2019;103(2):291-298. doi: 10.1097/TP.0000000000002384. PubMed PMID: 30119058; PubMed Central PMCID: PMCPMC6365242.

10 Guragain, B., Wei, Y., Zhang, H., Kahn-Krell, A., Ye, L., Walcott, G. P., Rogers, J. M. & Zhang, J. J. Implanted Human Cardiac Spheroids Electrically Couple With Infarcted Swine Myocardium. Circulation. 2024;149(23):1855-1857. Epub 20240603. doi: 10.1161/CIRCULATIONAHA.123.068568. PubMed PMID: 38829934; PubMed Central PMCID: PMCPMC11149905.

11 Chong, J. J., Yang, X., Don, C. W., Minami, E., Liu, Y. W., Weyers, J. J., Mahoney, W. M., Van Biber, B., Cook, S. M., Palpant, N. J., Gantz, J. A., Fugate, J. A., Muskheli, V., Gough, G. M., Vogel, K. W., Astley, C. A., Hotchkiss, C. E., Baldessari, A., Pabon, L., Reinecke, H., Gill, E. A., Nelson, V., Kiem, H. P., Laflamme, M. A. & Murry, C. E. Human embryonic-stem-cell-derived cardiomyocytes regenerate non-human primate hearts. Nature. 2014;510(7504):273-277. Epub 2014 Apr. 30. doi: 10.1038/nature13233. PubMed PMID: 24776797; PubMed Central PMCID: PMCPMC 4154594.

12 Cheng, Y. C., Hsieh, M. L., Lin, C. J., Chang, C. M. C., Huang, C. Y., Puntney, R., Wu Moy, A., Ting, C. Y., Herr Chan, D. Z., Nicholson, M. W., Lin, P. J., Chen, H. C., Kim, G. C., Zhang, J., Coonen, J., Basu, P., Simmons, H. A., Liu, Y. W., Hacker, T. A., Kamp, T. J. & Hsieh, P. C. H. Combined Treatment of Human Induced Pluripotent Stem Cell-Derived Cardiomyocytes and Endothelial Cells Regenerate the Infarcted Heart in Mice and Non-Human Primates. Circulation. 2023. Epub 2023 Sep. 21. doi: 10.1161/CIRCULATIONAHA.122.061736. PubMed PMID: 37732466.

13 Kobayashi, H., Tohyama, S., Ichimura, H., Ohashi, N., Chino, S., Soma, Y., Tani, H., Tanaka, Y., Yang, X., Shiba, N., Kadota, S., Haga, K., Moriwaki, T., Morita-Umei, Y., Umei, T. C., Sekine, O., Kishino, Y., Kanazawa, H., Kawagishi, H., Yamada, M., Narita, K., Naito, T., Seto, T., Kuwahara, K., Shiba, Y. & Fukuda, K. Regeneration of Nonhuman Primate Hearts With Human Induced Pluripotent Stem Cell-Derived Cardiac Spheroids. Circulation. 2024. Epub 2024 Apr. 26. doi: 10.1161/CIRCULATIONAHA.123.064876. PubMed PMID: 38666382.

14 Wu, K. H., Mo, X. M., Han, Z. C. & Zhou, B. Stem cell engraftment and survival in the ischemic heart. Ann Thorac Surg. 2011;92(5):1917-1925. doi: 10.1016/j.athoracsur.2011.07.012. PubMed PMID: 21955573.

15 Nguyen, P. K., Neofytou, E., Rhee, J. W. & Wu, J. C. Potential Strategies to Address the Major Clinical Barriers Facing Stem Cell Regenerative Therapy for Cardiovascular Disease: A Review. JAMA Cardiol. 2016;1(8):953-962. doi: 10.1001/jamacardio.2016.2750. PubMed PMID: 27579998; PubMed Central PMCID: PMCPMC5378463.

16 Weinberger, F., Breckwoldt, K., Pecha, S., Kelly, A., Geertz, B., Starbatty, J., Yorgan, T., Cheng, K. H., Lessmann, K., Stolen, T., Scherrer-Crosbie, M., Smith, G., Reichenspurner, H., Hansen, A. & Eschenhagen, T. Cardiac repair in guinea pigs with human engineered heart tissue from induced pluripotent stem cells. Science translational medicine. 2016;8(363):363ra148. Epub 2016 Nov. 4. doi: 10.1126/scitranslmed.aaf8781. PubMed PMID: 27807283.

17 Redd, M. A., Zeinstra, N., Qin, W., Wei, W., Martinson, A., Wang, Y., Wang, R. K., Murry, C. E. & Zheng, Y. Patterned human microvascular grafts enable rapid vascularization and increase perfusion in infarcted rat hearts. Nature communications. 2019;10(1):584. Epub 2019 Feb. 6. doi: 10.1038/s41467-019-08388-7. PubMed PMID: 30718840; PubMed Central PMCID: PMCPMC 6362250.

18 Gao, L., Gregorich, Z. R., Zhu, W., Mattapally, S., Oduk, Y., Lou, X., Kannappan, R., Borovjagin, A. V., Walcott, G. P., Pollard, A. E., Fast, V. G., Hu, X., Lloyd, S. G., Ge, Y. & Zhang, J. Large Cardiac Muscle Patches Engineered From Human Induced-Pluripotent Stem Cell-Derived Cardiac Cells Improve Recovery From Myocardial Infarction in Swine. Circulation. 2018;137(16):1712-1730. Epub 2017 Dec. 14. doi: 10.1161/CIRCULATIONAHA.117.030785. PubMed PMID: 29233823; PubMed Central PMCID: PMCPMC5903991.

19 Gerbin, K. A., Yang, X., Murry, C. E. & Coulombe, K. L. Enhanced Electrical Integration of Engineered Human Myocardium via Intramyocardial versus Epicardial Delivery in Infarcted Rat Hearts. PloS one. 2015; 10(7):e0131446. Epub 2015 Jul. 15. doi: 10.1371/journal. pone.0131446. PubMed PMID: 26161513; PubMed Central PMCID: PMC4498815.

20 Munarin, F., Kant, R. J., Rupert, C. E., Khoo, A. & Coulombe, K. L. K. Engineered human myocardium with local release of angiogenic proteins improves vascularization and cardiac function in injured rat hearts. Biomaterials. 2020;251:120033. Epub 2020 May 11. doi: 10.1016/j.biomaterials.2020.120033. PubMed PMID: 32388033.

21 Sugiura, T., Hibino, N., Breuer, C. K. & Shinoka, T. Tissue-engineered cardiac patch seeded with human induced pluripotent stem cell derived cardiomyocytes promoted the regeneration of host cardiomyocytes in a rat model. J Cardiothorac Surg. 2016;11(1):163. doi: 10.1186/s13019-016-0559-z. PubMed PMID: 27906085; PubMed Central PMCID: PMCPMC5131419.

22 Huang, N. F., Serpooshan, V., Morris, V. B., Sayed, N., Pardon, G., Abilez, O. J., Nakayama, K. H., Pruitt, B. L., Wu, S. M., Yoon, Y. S., Zhang, J. & Wu, J. C. Big bottlenecks in cardiovascular tissue engineering. Commun Biol. 2018; 1:199. Epub 2018 Nov. 28. doi: 10.1038/s42003-018-0202-8. PubMed PMID: 30480100; PubMed Central PMCID: PMCPMC6249300 declare no competing interests.

23 Gunter, J., Wolint, P., Bopp, A., Steiger, J., Cambria, E., Hoerstrup, S. P. & Emmert, M. Y. Microtissues in Cardiovascular Medicine: Regenerative Potential Based on a 3D Microenvironment. Stem Cells Int. 2016; 2016:9098523. Epub 2016 Apr. 14. doi: 10.1155/2016/9098523. PubMed PMID: 27073399; PubMed Central PMCID: PMCPMC4814701.

24 Coyle, R. C., Barrs, R. W., Richards, D. J., Ladd, E. P., Menick, D. R. & Mei, Y. Targeting HIF-alpha for robust prevascularization of human cardiac organoids. Journal of tissue engineering and regenerative medicine. 2021;15(2):189-202. Epub 2021 Apr. 20. doi: 10.1002/term.3165. PubMed PMID: 33868541; PubMed Central PMCID: PMCPMC8049092.

25 Richards, D. J., Coyle, R. C., Tan, Y., Jia, J., Wong, K., Toomer, K., Menick, D. R. & Mei, Y. Inspiration from heart development: Biomimetic development of functional human cardiac organoids. Biomaterials. 2017;142:112-123. doi: 10.1016/j.biomaterials.2017.07.021. PubMed PMID: 28732246; PubMed Central PMCID: PMCPMC5562398.

26 Zhao, X., Chen, H., Xiao, D., Yang, H., Itzhaki, I., Qin, X., Chour, T., Aguirre, A., Lehmann, K., Kim, Y., Shukla, P., Holmstrom, A., Zhang, J. Z., Zhuge, Y., Ndoye, B. C., Zhao, M., Neofytou, E., Zimmermann, W. H., Jain, M. & Wu, J. C. Comparison of Non-human Primate versus Human Induced Pluripotent Stem Cell-Derived Cardiomyocytes for Treatment of Myocardial Infarction. Stem cell reports. 2018; 10(2):422-435. Epub 2018 Feb. 6. doi: 10.1016/j. stemcr.2018.01.002. PubMed PMID: 29398480; PubMed Central PMCID: PMCPMC5830958.

27 Fernandes, S., Chong, J. J., Paige, S. L., Iwata, M., Torok-Storb, B., Keller, G., Reinecke, H. & Murry, C. E. Comparison of Human Embryonic Stem Cell-Derived Cardiomyocytes, Cardiovascular Progenitors, and Bone Marrow Mononuclear Cells for Cardiac Repair. Stem cell reports. 2015(5):753-762. Epub 2015 Nov. 27. doi: 10.1016/j. stemcr.2015.09.011. PubMed PMID: 26607951; PubMed Central PMCID: PMCPMC4649260

28 Kawamura, T., Miyagawa, S., Fukushima, S., Maeda, A., Kashiyama, N., Kawamura, A., Miki, K., Okita, K., Yoshida, Y., Shiina, T., Ogasawara, K., Miyagawa, S., Toda, K., Okuyama, H. & Sawa, Y. Cardiomyocytes Derived from MHC-Homozygous Induced Pluripotent Stem Cells Exhibit Reduced Allogeneic Immunogenicity in MHC-Matched Non-human Primates. Stem cell reports. 2016;6(3):312-320. Epub 2016 Feb. 26. doi: 10.1016/j.stemcr.2016.01.012. PubMed PMID: 26905198; PubMed Central PMCID: PMCPMC4788782.

29 Giacomelli, E., Meraviglia, V., Campostrini, G., Cochrane, A., Cao, X., van Helden, R. W. J., Krotenberg Garcia, A., Mircea, M., Kostidis, S., Davis, R. P., van Meer, B. J., Jost, C. R., Koster, A. J., Mei, H., Miguez, D. G., Mulder, A. A., Ledesma-Terron, M., Pompilio, G., Sala, L., Salvatori, D. C. F., Slieker, R. C., Sommariva, E., de Vries, A. A. F., Giera, M., Semrau, S., Tertoolen, L. G. J., Orlova, V. V., Bellin, M. & Mummery, C. L. Human-iPSC-Derived Cardiac Stromal Cells Enhance Maturation in 3D Cardiac Microtissues and Reveal Non-cardiomyocyte Contributions to Heart Disease. Cell stem cell. 2020; 26(6):862-879 e811. Epub 2020 May 28. doi: 10.1016/j.stem.2020.05.004. PubMed PMID: 32459996.

30 Campostrini, G., Meraviglia, V., Giacomelli, E., van Helden, R. W. J., Yiangou, L., Davis, R. P., Bellin, M., Orlova, V. V. & Mummery, C. L. Generation, functional analysis and applications of isogenic three-dimensional self-aggregating cardiac microtissues from human pluripotent stem cells. Nat Protoc. 2021;16(4):2213-2256. Epub 2021 Mar. 28. doi: 10.1038/s 41596-021-00497-2. PubMed PMID: 33772245.

31 Dvir, T., Kedem, A., Ruvinov, E., Levy, O., Freeman, I., Landa, N., Holbova, R., Feinberg, M. S., Dror, S., Etzion, Y., Leor, J. & Cohen, S. Prevascularization of cardiac patch on the omentum improves its therapeutic outcome. Proceedings of the National Academy of Sciences of the United States of America. 2009;106(35):14990-14995. Epub 2009 Aug. 27. doi: 10.1073/pnas.0812242106. PubMed PMID: 19706385; PubMed Central PMCID: PMC2736451.

32 Kerr, C. M., Silver, S. E., Choi, Y. S., Floy, M. E., Bradshaw, A. D., Cho, S. W., Palecek, S. P. & Mei, Y. Decellularized heart extracellular matrix alleviates activation of hiPSC-derived cardiac fibroblasts. Bioact Mater. 2024;31:463-474. Epub 2023 Sep. 13. doi: 10.1016/j. bioactmat.2023.08.023. PubMed PMID: 37701451; PubMed Central PMCID: PMCPMC10493503.

33 Lian, X., Zhang, J., Azarin, S. M., Zhu, K., Hazeltine, L. B., Bao, X., Hsiao, C., Kamp, T. J. & Palecek, S. P. Directed cardiomyocyte differentiation from human pluripotent stem cells by modulating Wnt/beta-catenin signaling under fully defined conditions. Nature protocols. 2013;8(1):162-175. Epub 2012 Dec. 22. doi: 10.1038/nprot.2012.150. PubMed PMID: 23257984; PubMed Central PMCID: PMCPMC 3612968.

34 Tohyama, S., Hattori, F., Sano, M., Hishiki, T., Nagahata, Y., Matsuura, T., Hashimoto, H., Suzuki, T., Yamashita, H., Satoh, Y., Egashira, T., Seki, T., Muraoka, N., Yamakawa, H., Ohgino, Y., Tanaka, T., Yoichi, M., Yuasa, S., Murata, M., Suematsu, M. & Fukuda, K. Distinct metabolic flow enables large-scale purification of mouse and human pluripotent stem cell-derived cardiomyocytes. Cell Stem Cell. 2013;12(1):127-137. doi: 10.1016/j. stem.2012.09.013. PubMed PMID: 23168164.

35 Maas, R. G. C., Lee, S., Harakalova, M., Snijders Blok, C. J. B., Goodyer, W. R., Hjortnaes, J., Doevendans, P., Van Laake, L. W., van der Velden, J., Asselbergs, F. W., Wu, J. C., Sluijter, J. P. G., Wu, S. M. & Buikema, J. W. Massive expansion and cryopreservation of functional human induced pluripotent stem cell-derived cardiomyocytes. STAR Protoc. 2021;2(1):100334. Epub 2021 Feb. 23. doi: 10.1016/j.xpro.2021.100334. PubMed PMID: 33615277; PubMed Central PMCID: PMCPMC7881265.

36 Buikema, J. W., Lee, S., Goodyer, W. R., Maas, R. G., Chirikian, O., Li, G., Miao, Y., Paige, S. L., Lee, D., Wu, H., Paik, D. T., Rhee, S., Tian, L., Galdos, F. X., Puluca, N., Beyersdorf, B., Hu, J., Beck, A., Venkamatran, S., Swami, S., Wijnker, P., Schuldt, M., Dorsch, L. M., van Mil, A., Red-Horse, K., Wu, J. Y., Geisen, C., Hesse, M., Serpooshan, V., Jovinge, S., Fleischmann, B. K., Doevendans, P. A., van der Velden, J., Garcia, K. C., Wu, J. C., Sluijter, J. P. G. & Wu, S. M. Wnt Activation and Reduced Cell-Cell Contact Synergistically Induce Massive Expansion of Functional Human iPSC-Derived Cardiomyocytes. Cell stem cell. 2020;27(1):50-63e55. Epub 2020 Jul. 4. doi: 10.1016/j.stem.2020.06.001. PubMed PMID: 32619518; PubMed Central PMCID: PMCPMC7334437.

37 Bao, X., Lian, X., Hacker, T. A., Schmuck, E. G., Qian, T., Bhute, V. J., Han, T., Shi, M., Drowley, L., Plowright, A., Wang, Q. D., Goumans, M. J. & Palecek, S. P. Long-term self-renewing human epicardial cells generated from pluripotent stem cells under defined xeno-free conditions. Nat Biomed Eng. 2016; 1. Epub 2016 Jan. 1. doi: 10.1038/s41551-016-0003. PubMed PMID: 28462012; PubMed Central PMCID: PMCPMC 5408455.

38 Bao, X., Lian, X., Qian, T., Bhute, V. J., Han, T. & Palecek, S. P. Directed differentiation and long-term maintenance of epicardial cells derived from human pluripotent stem cells under fully defined conditions. Nat Protoc. 2017;12(9):1890-1900. Epub 2017 Aug. 18. doi: 10.1038/nprot.2017.080. PubMed PMID: 28817124; PubMed Central PMCID: PMCPMC5630264.

39 Jin, Y., Kim, H., Min, S., Choi, Y. S., Seo, S. J., Jeong, E., Kim, S. K., Lee, H. A., Jo, S. H., Park, J. H., Park, B. W., Sim, W. S., Kim, J. J., Ban, K., Kim, Y. G., Park, H. J. & Cho, S. W. Three-dimensional heart extracellular matrix enhances chemically induced direct cardiac reprogramming. Sci Adv. 2022;8(50):eabn5768. Epub 2022 Dec. 15. doi: 10.1126/sciadv.abn5768. PubMed PMID: 36516259; PubMed Central PMCID: PMCPMC9750148.

40 Cho, H., Macklin, B. L., Lin, Y. Y., Zhou, L., Lai, M. J., Lee, G., Gerecht, S. & Duh, E. J. iPSC-derived endothelial cell response to hypoxia via SDF1a/CXCR4 axis facilitates incorporation to revascularize ischemic retina. JCI Insight. 2020;5(6). Epub 2020 Mar. 28. doi: 10.1172/jci.insight.131828. PubMed PMID: 32213707; PubMed Central PMCID: PMCPMC7213794.

41 Smith, Q., Macklin, B., Chan, X. Y., Jones, H., Trempel, M., Yoder, M. C. & Gerecht, S. Differential HDAC6 Activity Modulates Ciliogenesis and Subsequent Mechanosensing of Endothelial Cells Derived from Pluripotent Stem Cells. Cell Rep. 2018;24(4):895-908 e896. Epub 2018 Jul. 26. doi: 10.1016/j.celrep.2018.06.083. PubMed PMID: 30044986.

42 Bao, X., Lian, X. & Palecek, S. P. Directed Endothelial Progenitor Differentiation from Human Pluripotent Stem Cells Via Wnt Activation Under Defined Conditions. Methods Mol Biol. 2016;1481:183-196. Epub 2016 Sep. 4. doi: 10.1007/978-1-4939-6393-5_17. PubMed PMID: 27590162; PubMed Central PMCID: PMCPMC 5075968.

43 Nishihara, H., Gastfriend, B. D., Soldati, S., Perriot, S., Mathias, A., Sano, Y., Shimizu, F., Gosselet, F., Kanda, T., Palecek, S. P., Du Pasquier, R., Shusta, E. V. & Engelhardt, B. Advancing human induced pluripotent stem cell-derived blood-brain barrier models for studying immune cell interactions. FASEB J. 2020;34(12):16693-16715. Epub 2020 Oct. 31. doi: 10.1096/fj.202001507RR. PubMed PMID: 33124083; PubMed Central PMCID: PMCPMC7686106.

44 Faal, T., Phan, D. T. T., Davtyan, H., Scarfone, V. M., Varady, E., Blurton-Jones, M., Hughes, C. C. W. & Inlay, M. A. Induction of Mesoderm and Neural Crest-Derived Pericytes from Human Pluripotent Stem Cells to Study Blood-Brain Barrier Interactions. Stem cell reports. 2019; 12(3):451-460. Epub 2019 Feb. 13. doi: 10.1016/j.stemcr.2019.01.005. PubMed PMID: 30745035; PubMed Central PMCID: PMCPMC6409424.

45 Richards, D. J., Li, Y., Kerr, C. M., Yao, J., Beeson, G. C., Coyle, R. C., Chen, X., Jia, J., Damon, B., Wilson, R., Starr Hazard, E., Hardiman, G., Menick, D. R., Beeson, C. C., Yao, H., Ye, T. & Mei, Y. Human cardiac organoids for the modelling of myocardial infarction and drug cardiotoxicity. Nat Biomed Eng. 2020;4(4):446-462. doi: 10.1038/s41551-020-0539-4. PubMed PMID: 32284552; PubMed Central PMCID: PMCPMC7422941.

46 Lian, X., Hsiao, C., Wilson, G., Zhu, K., Hazeltine, L. B., Azarin, S. M., Raval, K. K., Zhang, J., Kamp, T. J. & Palecek, S. P. Robust cardiomyocyte differentiation from human pluripotent stem cells via temporal modulation of canonical Wnt signaling. Proc Natl Acad Sci USA. 2012;109(27):E1848-1857. doi: 10.1073/pnas.1200250109. PubMed PMID: 22645348; PubMed Central PMCID: PMCPMC 3390875.

47 Aghajanian, H., Kimura, T., Rurik, J. G., Hancock, A. S., Leibowitz, M. S., Li, L., Scholler, J., Monslow, J., Lo, A., Han, W., Wang, T., Bedi, K., Morley, M. P., Linares Saldana, R. A., Bolar, N. A., McDaid, K., Assenmacher, C. A., Smith, C. L., Wirth, D., June, C. H., Margulies, K. B., Jain, R., Pure, E., Albelda, S. M. & Epstein, J. A. Targeting cardiac fibrosis with engineered T cells. Nature. 2019;573(7774):430-433. Epub 2019/09/13. doi: 10.1038/s41586-019-1546-z. PubMed PMID: 31511695; PubMed Central PMCID: PMCPMC6752964.

48 Floy, M. E., Givens, S. E., Matthys, O. B., Mateyka, T. D., Kerr, C. M., Steinberg, A. B., Silva, A. C., Zhang, J., Mei, Y., Ogle, B. M., McDevitt, T. C., Kamp, T. J. & Palecek, S. P. Developmental lineage of human pluripotent stem cell-derived cardiac fibroblasts affects their functional phenotype. FASEB J. 2021;35(9):e21799. Epub 2021 Aug. 3. doi: 10.1096/fj. 202100523R. PubMed PMID: 34339055; PubMed Central PMCID: PMCPMC8349112.

49 Zhang, H., Tian, L., Shen, M., Tu, C., Wu, H., Gu, M., Paik, D. T. & Wu, J. C.

Generation of Quiescent Cardiac Fibroblasts From Human Induced Pluripotent Stem Cells for In Vitro Modeling of Cardiac Fibrosis. Circulation research. 2019;125(5):552-566. Epub 2019/07/11. doi: 10.1161/CIRCRESAHA.119.315491. PubMed PMID: 31288631; PubMed Central PMCID: PMCPMC 6768436.

50 Lian, X., Bao, X., Al-Ahmad, A., Liu, J., Wu, Y., Dong, W., Dunn, K. K., Shusta, E. V. & Palecek, S. P. Efficient differentiation of human pluripotent stem cells to endothelial progenitors via small-molecule activation of WNT signaling. Stem cell reports. 2014;3(5):804-816. Epub 2014 Nov. 25. doi: 10.1016/j.stemcr.2014.09.005. PubMed PMID: 25418725; PubMed Central PMCID: PMCPMC 4235141.

51 James, D., Nam, H. S., Seandel, M., Nolan, D., Janovitz, T., Tomishima, M., Studer, L., Lee, G., Lyden, D., Benezra, R., Zaninovic, N., Rosenwaks, Z., Rabbany, S. Y. & Rafii, S. Expansion and maintenance of human embryonic stem cell-derived endothelial cells by TGFbeta inhibition is Id1 dependent. Nat Biotechnol. 2010;28(2):161-166. Epub 2010 Jan. 19. doi: 10.1038/nbt.1605. PubMed PMID: 20081865; PubMed Central PMCID: PMCPMC2931334

52 Dewing, J. M., Saunders, V., O'Kelly, I. & Wilson, D. I. Defining cardiac cell populations and relative cellular composition of the early fetal human heart. PLOS One. 2022;17(11):e0259477. Epub 2022 Dec. 1. doi: 10.1371/journal.pone.0259477. PubMed PMID: 36449524; PubMed Central PMCID: PMCPMC 9710754 interests: IO is a paid employee of Immunocore. There are no patents, products in development or marketed products associated with this research to declare. This does not alter our adherence to PLOS ONE policies on sharing data and materials.

53 Nees, S., Weiss, D. R., Senftl, A., Knott, M., Forch, S., Schnurr, M., Weyrich, P. & Juchem, G. Isolation, bulk cultivation, and characterization of coronary microvascular pericytes: the second most frequent myocardial cell type in vitro. Am J Physiol Heart Circ Physiol. 2012;302(1):H69-84. Epub 2011 Nov. 1. doi: 10.1152/ajpheart.00359.2011. PubMed PMID: 22037185.

54 Colliva, A., Braga, L., Giacca, M. & Zacchigna, S. Endothelial cell-cardiomyocyte crosstalk in heart development and disease. The Journal of physiology. 2020;598(14):2923-2939. Epub 2019 Mar. 1. doi: 10.1113/JP276758. PubMed PMID: 30816576; PubMed Central PMCID: PMCPMC 7496632.

55 Karbassi, E., Fenix, A., Marchiano, S., Muraoka, N., Nakamura, K., Yang, X. & Murry, C. E. Cardiomyocyte maturation: advances in knowledge and implications for regenerative medicine. Nature reviews. Cardiology. 2020;17(6):341-359. Epub 2020 Feb. 6. doi: 10.1038/s41569-019-0331-x. PubMed PMID: 32015528; PubMed Central PMCID: PMCPMC7239749.

56 Burchfield, J. S., Xie, M. & Hill, J. A. Pathological ventricular remodeling: mechanisms: part 1 of 2. Circulation. 2013;128(4):388-400. Epub 2013 Jul. 24. doi: 10.1161/CIRCULATIONAHA.113.001878. PubMed PMID: 23877061; PubMed Central PMCID: PMCPMC3801217.

57 Avolio, E. & Madeddu, P. Discovering cardiac pericyte biology: From physiopathological mechanisms to potential therapeutic applications in ischemic heart disease. Vascul Pharmacol. 2016;86:53-63. Epub 2016 Jun. 9. doi: 10.1016/j. vph.2016.05.009. PubMed PMID: 27268036.

58 Armulik, A., Abramsson, A. & Betsholtz, C. Endothelial/pericyte interactions. Circulation research. 2005;97(6):512-523. Epub 2005 Sep. 17. doi: 10.1161/01.RES.0000182903.16652.d7. PubMed PMID: 16166562.Paik, J. H., Skoura, A., Chae, S. S., Cowan, A. E., Han, D. K., Proia, R. L. & Hla, T.

59 Sphingosine 1-phosphate receptor regulation of N-cadherin mediates vascular stabilization. Genes Dev. 2004;18(19):2392-2403. Epub 2004 Sep. 17. doi: 10.1101/gad. 1227804. PubMed PMID: 15371328; PubMed Central PMCID: PMCPMC522989.

60 Gerhardt, H., Wolburg, H. & Redies, C. N-cadherin mediates pericytic-endothelial interaction during brain angiogenesis in the chicken. Dev Dyn. 2000;218(3):472-479. Epub 2000 Jul. 6. doi: 10.1002/1097-0177(200007)218:3<472::AID-DVDY1008>3.0.CO; 2-#. PubMed PMID: 10878612.

61 Vantler, M., Huntgeburth, M., Caglayan, E., Ten Freyhaus, H., Schnabel, P. & Rosenkranz, S. PI3-kinase/Akt-dependent antiapoptotic signaling by the PDGF alpha receptor is negatively regulated by Src family kinases. FEBS Lett. 2006; 580(30):6769-6776. Epub 2006 Dec. 5. doi: 10.1016/j.febslet.2006.11.034. PubMed PMID: 17141222.

62 Vantler, M., Karikkineth, B. C., Naito, H., Tiburcy, M., Didie, M., Nose, M., Rosenkranz, S. & Zimmermann, W. H. PDGF-BB protects cardiomyocytes from apoptosis and improves contractile function of engineered heart tissue. J Mol Cell Cardiol. 2010;48(6):1316-1323. Epub 2010 Mar. 24. doi: 10.1016/j.yjmcc.2010.03.008. PubMed PMID: 20307544.

63 Jin, S., Guerrero-Juarez, C. F., Zhang, L., Chang, I., Ramos, R., Kuan, C. H., Myung, P., Plikus, M. V. & Nie, Q. Inference and analysis of cell-cell communication using CellChat. Nature communications. 2021; 12(1):1088. Epub 20210217. doi: 10.1038/s41467-021-21246-9. PubMed PMID: 33597522; PubMed Central PMCID: PMCPMC7889871.

64 Farah, E. N., Hu, R. K., Kern, C., Zhang, Q., Lu, T. Y., Ma, Q., Tran, S., Zhang, B., Carlin, D., Monell, A., Blair, A. P., Wang, Z., Eschbach, J., Li, B., Destici, E., Ren, B., Evans, S. M., Chen, S., Zhu, Q. & Chi, N. C. Spatially organized cellular communities form the developing human heart. Nature. 2024;627(8005):854-864. Epub 2024 Mar. 14. doi: 10.1038/s41586-024-07171-z. PubMed PMID: 38480880; PubMed Central PMCID: PMCPMC10972757 Epigenome Technologies. All other authors have no conflicts of interest.

65 Lin, Z., Garbern, J. C., Liu, R., Li, Q., Mancheno Juncosa, E., Elwell, H. L. T., Sokol, M., Aoyama, J., Deumer, U.S., Hsiao, E., Sheng, H., Lee, R. T. & Liu, J. Tissue-embedded stretchable nanoelectronics reveal endothelial cell-mediated electrical maturation of human 3D cardiac microtissues. Sci Adv. 2023;9(10):eade8513. Epub 2023 Mar. 9. doi: 10.1126/sciadv.ade8513. PubMed PMID: 36888704; PubMed Central PMCID: PMCPMC9995081.

### Example 2

### Preparation of Pre-vascularized, Nanowired Cardiac Organoids

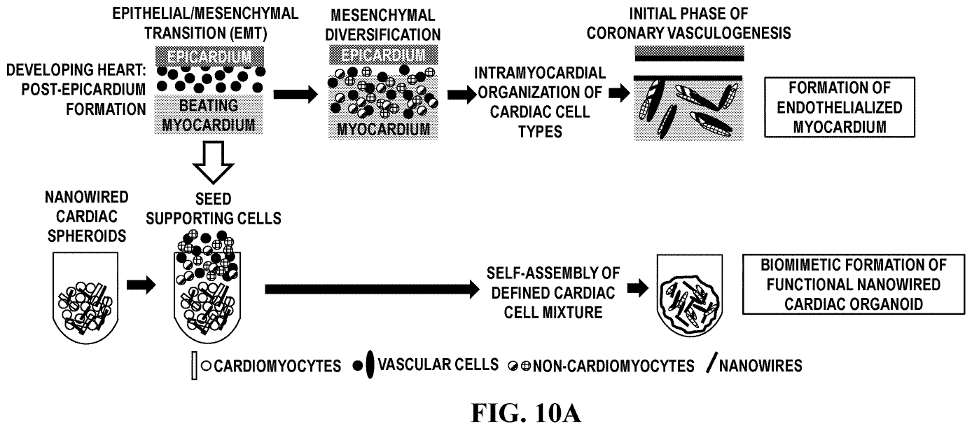

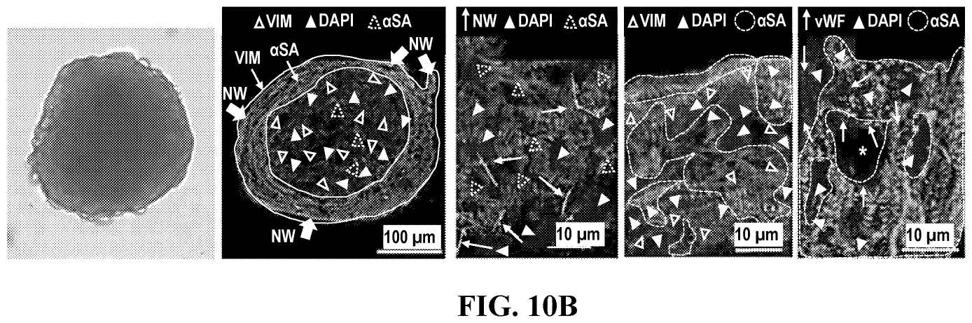

Pre-vascularized, nanowired human cardiac organoids were prepared as depicted in FIGS. 10A-10B.

### Example 3

### Implantation of Cardiac Organoids and Nanowired Cardiac Organoids

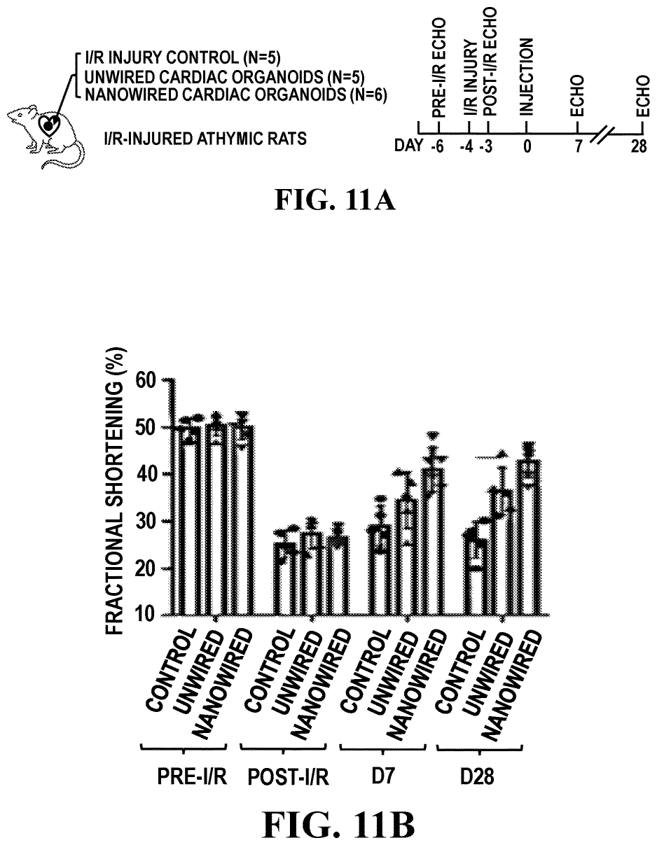

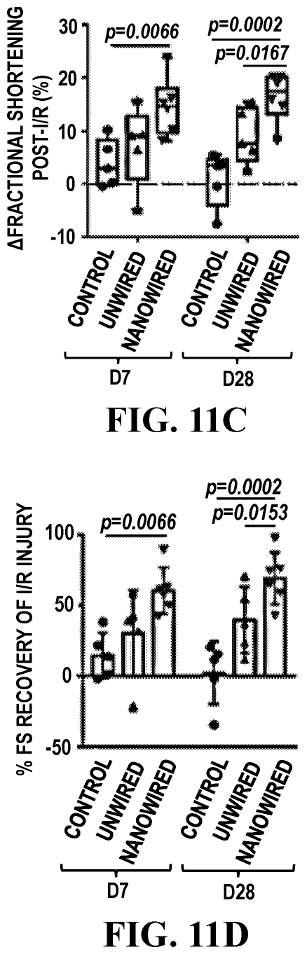

A rat myocardial ischemia/reperfusion (I/R) model was prepared by implanting, via intramyocardial injection, unwired cardiac organoids (n=5) and nanowired cardiac organoids (n=6) into I/R-injured athymic rats (FIG. 11A). The implantations resulted in about 1 million total cells per rat, including about 0.5 million hPSC-CMs per rat, implanted during organoid injections. This method differs from the current state of the art, which methods include implanting 10 million single hPSC-CMs per rat with about 40% fractional shortening (FS) recovery at Day 28. State-of-the-art methods use about 20-fold less hPSC-CMs per rat as compared to the method described herein.

As shown in FIGS. 11B-11D, nanowired organoids provide improved functional recovery of I/R injured rat hearts as compared to state-of-the-art methods.

### Example 4

### Preparation and Implantation of Hypoimmune Organoids

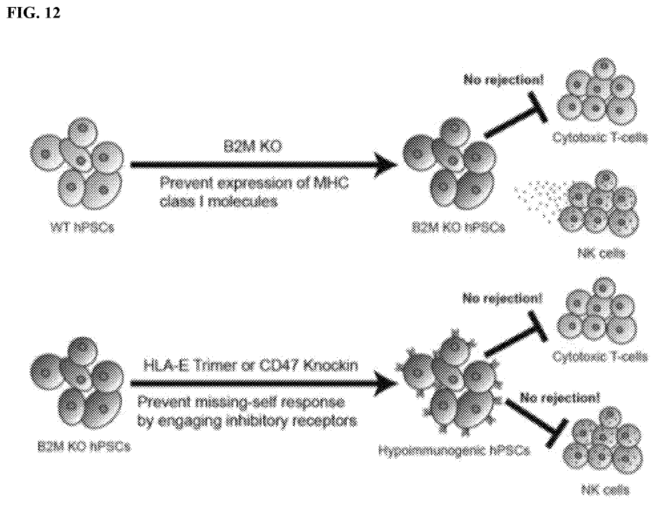

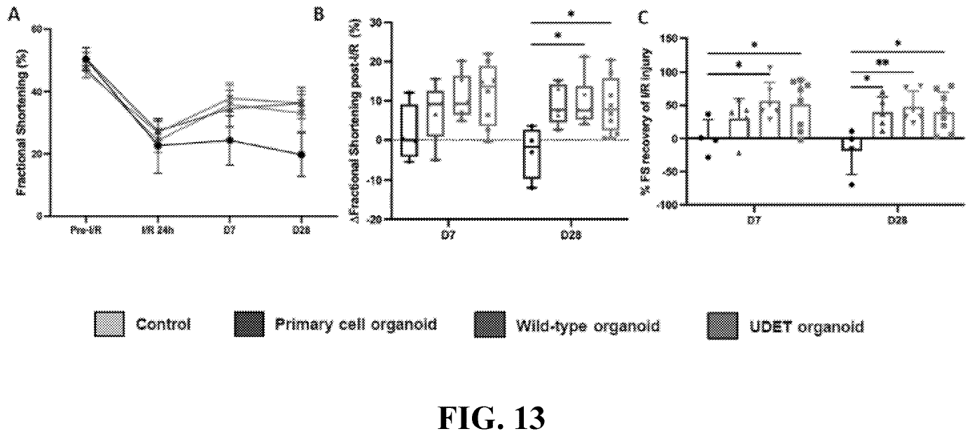

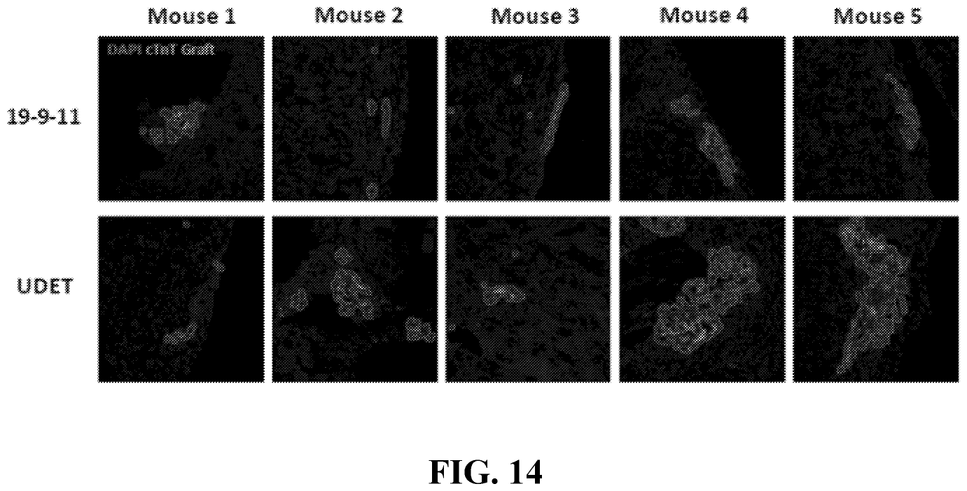

Strategies were employed to establish hypoimmune organoids, as depicted in FIG. 12. The hypoimmune organoids, which contained Universal Donor HLA-E Trimer (“UDET”) hPSC cardiac cells, were implanted (e.g., via intramyocardial injection) to ischemia/reperfusion (I/R) injured hearts in I/R-injured athymic rats. Isogenic, wild-type human cardiac organoids (i.e., comprising 19-9-11 hiPSCs) were also implanted in I/R-injured athymic rats. UDET cardiac organoids were shown to recover functions in infarcted hearts, particularly with a capacity comparable to the isogenic, wild-type human cardiac organoids, as shown in FIG. 13, Panels A-C.

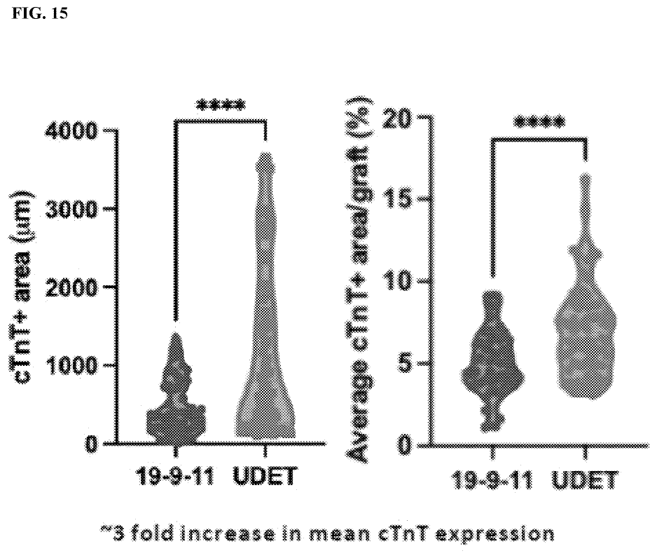

In addition, UDET cardiac organoids were implanted in allogeneic humanized mice, as were isogenic, wild-type cardiac organoids. UDET cardiac organoids were shown to promote hPSC-CM survival and retention in the mice, as shown in FIGS. 14 and 15. In addition, pericytes in the organoids expressed PDL1 that can promote immune tolerance for the organoids.

The foregoing is illustrative of the present invention, and is not to be construed as limiting thereof. The invention is defined by the following claims, with equivalents of the claims to be included therein.

1. An isogenic three-dimensional (3D) cardiac organoid, comprising four different cardiac cell types that comprise cardiomyocytes (CMs) and nonmyocytes, wherein the cardiomyocytes and nonmyocytes are derived from isogenic pluripotent stem cells (iPSCs).

2. The isogenic 3D cardiac organoid of claim 1, wherein the nonmyocytes comprise pericytes (PCs).

3. The isogenic 3D cardiac organoid of claim 1, wherein the cardiomyocytes and nonmyocytes are derived from isogenic autologous pluripotent stem cells (PSCs).

4. The isogenic 3D cardiac organoid of claim 1, wherein the cardiomyocytes and nonmyocytes are derived from isogenic allogeneic pluripotent stem cells (PSCs).

5. The isogenic 3D cardiac organoid of claim 1, wherein the cardiomyocytes comprise induced pluripotent stem cell-derived cardiomyocytes (iPSC-CMs), cardiac progenitor cells, primary cardiomyocytes, or any combination thereof.

6. The isogenic 3D cardiac organoid of claim 1, wherein the nonmyocytes comprise fibroblasts (FBs), endothelial cells (ECs), pericytes (PCs) and/or mesenchymal stem cells (MSCs).

7. The isogenic 3D cardiac organoid of claim 1, wherein the organoid comprises iPSC-CMs, iPSC-derived fibroblasts (iPSC-Fbs), iPSC-derived endothelial cells (iPSC-ECs), and iPSC-derived pericytes (iPSC-PCs).

8. The isogenic 3D cardiac organoid of claim 1, wherein the 3D cardiac organoid comprises an average diameter of about 400 microns (μm) or less.

9. The isogenic 3D cardiac organoid of claim 1, wherein the cardiomyocytes and/or nonmyocytes and/or the source isogenic pluripotent stem cells (iPSCs) comprise a hypoimmune and/or immune evading modification.

10. (canceled)

11. The isogenic 3D cardiac organoid of claim 9, wherein the hypoimmune and/or immune evading modification is a beta-2 microglobulin (β2M) mutation rendering β2M non-functional.

12. The isogenic 3D cardiac organoid of claim 1, wherein the organoid further comprises nanowires.

13. The isogenic 3D cardiac organoid of claim 1, wherein the organoid beats spontaneously.

14. The isogenic 3D cardiac organoid of claim 1, wherein the cardiomyocytes and nonmyocytes are present in a ratio of about 95:5 to about 5:95 of cardiomyocytes to nonmyocytes.

15. The isogenic 3D cardiac organoid of claim 1, wherein the nonmyocytes comprise fibroblasts in amount of about 15% to 50% based on the total number of nonmyocytes, endothelial cells in an amount of about 25% to about 75% based on the total number of nonmyocytes, and pericytes in an amount of about 5% to about 30% based on the total number of nonmyocytes.

16. The isogenic 3D cardiac organoid of claim 1, wherein the cardiomyocytes and/or the nonmyocytes are from a human.

17. (canceled)

18. The isogenic 3D cardiac organoid of claim 1, wherein the organoid is devoid of a hydrogel.

19. A method of making an isogenic 3D cardiac organoid, the method comprising:
culturing isogenic cardiomyocytes derived from source isogenic pluripotent stem cells (iPSCs) with isogenic nonmyocytes derived from source isogenic pluripotent stem cells (iPSCs) for about 1 day to about 10 days to form a self-assembled 3D cardiac organoid under normoxic conditions;
thereby generating the 3D cardiac organoid.

20-31. (canceled)

32. A method of treating a subject in need thereof, comprising administering to the subject a therapeutically effective amount of an isogenic three-dimensional (3D) cardiac organoid, wherein the isogenic 3D cardiac organoid comprises four different cardiac cell types that comprise cardiomyocytes (CMs) and nonmyocytes, wherein the cardiomyocytes and nonmyocytes are derived from isogenic pluripotent stem cells (iPSCs).

33. The method of claim 32, wherein the administered isogenic 3D cardiac organoid has enhanced engraftment in the subject as compared to a control.

34. (canceled)

35. The method of claim 32, wherein the therapeutically effective amount of the isogenic 3D cardiac organoid is administered to the subject by implanting the therapeutically effective amount of the isogenic 3D cardiac organoid in the subject.

36-37. (canceled)
